(19) United States
(12) Patent Application Publication
(10) Pub. No.: US 2008/0292587 A1
(43) Pub. Date: Nov. 27, 2008

(54) OXIMYL DIPEPTIDE HEPATITIS C PROTEASE INHIBITORS

(76) Inventors: Ying Sun, Waltham, MA (US); Yat Sun Or, Watertown, MA (US); Zhe Wang, Hockessin, DE (US)

Correspondence Address: ELMORE PATENT LAW GROUP, PC, 515 Groton Road, Unit 1R, Westford, MA, 01886, US

(21) Appl. No.: 12/108,659

(22) Filed: Apr. 24, 2008

Related U.S. Application Data
(60) Provisional application No. 60/914,181, filed on Apr. 26, 2007.

(51) Int. Cl. A61K 38/05; C07D 207/12; C07D 409/12; A61P 31/12; A61K 38/21; C07D 405/12

(52) U.S. Cl. 424/85.6; 548/556; 548/527; 548/517; 548/525; 514/19; 424/85.7

(57) ABSTRACT

The present invention discloses compounds of formulae I, or pharmaceutically acceptable salts, esters, or prodrugs thereof:

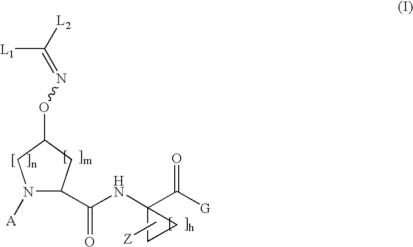

which inhibit serine protease activity, particularly the activity of hepatitis C virus (HCV) NS3-NS4A protease. Consequently, the compounds of the present invention interfere with the life cycle of the hepatitis C virus and are also useful as antiviral agents. The present invention further relates to pharmaceutical compositions comprising the aforementioned compounds for administration to a subject suffering from HCV infection. The invention also relates to methods of treating an HCV infection in a subject by administering a pharmaceutical composition comprising the compounds of the present invention.

### CROSS-REFERENCE TO RELATED APPLICATIONS

This application claims benefit of U.S. provisional application 60/914,181 filed Apr. 26, 2007, the entire content of which is herein incorporated by reference.

### TECHNICAL FIELD

The present invention relates to novel HCV derivatives having activity against hepatitis C virus (HCV) and useful in the treatment of HCV infections. More particularly, the invention relates to HCV compounds, compositions containing such compounds and methods for using the same, as well as processes for making such compounds.

### BACKGROUND OF THE INVENTION

HCV is the principal cause of non-A, non-B hepatitis and is an increasingly severe public health problem both in the developed and developing world. It is estimated that the virus infects over 200 million people worldwide, surpassing the number of individuals infected with the human immunodeficiency virus (HIV) by nearly five fold. HCV infected patients, due to the high percentage of individuals inflicted with chronic infections, are at an elevated risk of developing cirrhosis of the liver, subsequent hepatocellular carcinoma and terminal liver disease. HCV is the most prevalent cause of hepatocellular cancer and cause of patients requiring liver transplantations in the western world.

There are considerable barriers to the development of anti-HCV therapeutics, which include, but are not limited to, the persistence of the virus, the genetic diversity of the virus during replication in the host, the high incident rate of the virus developing drug-resistant mutants, and the lack of reproducible infectious culture systems and small-animal models for HCV replication and pathogenesis. In a majority of cases, given the mild course of the infection and the complex biology of the liver, careful consideration must be given to antiviral drugs, which are likely to have significant side effects.

Only two approved therapies for HCV infection are currently available. The original treatment regimen generally involves a 3-12 month course of intravenous interferon-alpha. (IFN-α), while a new approved second-generation treatment involves co-treatment with IFN-α and the general antiviral nucleoside mimics like ribavirin. Both of these treatments suffer from interferon-related side effects as well as low efficacy against HCV infections. There exists a need for the development of effective antiviral agents for treatment of HCV infection due to the poor tolerability and disappointing efficacy of existing therapies.

In a patient population where the majority of individuals are chronically infected and asymptomatic and the prognoses are unknown, an effective drug must possess significantly fewer side effects than the currently available treatments. The hepatitis C non-structural protein-3 (NS3) is a proteolytic enzyme required for processing of the viral polyprotein and consequently viral replication. Despite the huge number of viral variants associated with HCV infection, the active site of the NS3 protease remains highly conserved thus making its inhibition an attractive mode of intervention. Recent success in the treatment of HIV with protease inhibitors supports the concept that the inhibition of NS3 is a key target in the battle against HCV.

HCV is a flaviridae type RNA virus. The HCV genome is enveloped and contains a single strand RNA molecule composed of circa 9600 base pairs. It encodes a polypeptide comprised of approximately 3010 amino acids.

The HCV polyprotein is processed by viral and host peptidase into 10 discreet peptides which serve a variety of functions. There are three structural proteins, C, E1 and E2. The P7 protein is of unknown function and is comprised of a highly variable sequence. There are six non-structural proteins. NS2 is a zinc-dependent metalloproteinase that functions in conjunction with a portion of the NS3 protein. NS3 incorporates two catalytic functions (separate from its association with NS2): a serine protease at the N-terminal end, which requires NS4A as a cofactor, and an ATP-ase-dependent helicase function at the carboxyl terminus. NS4A is a tightly associated but non-covalent cofactor of the serine protease.

The NS34A protease is responsible for cleaving four sites on the viral polyprotein. The NS3-NS4A cleavage is autocatalytic, occurring in cis. The remaining three hydrolyses, NS4A-NS4B, NS4B-NS5A and NS5A-NS5B all occur in trans. NS3 is a serine protease which is structurally classified as a chymotrypsin-like protease. While the NS serine protease possesses proteolytic activity by itself, the HCV protease enzyme is not an efficient enzyme in terms of catalyzing polyprotein cleavage. It has been shown that a central hydrophobic region of the NS4A protein is required for this enhancement. The complex formation of the NS3 protein with NS4A seems necessary to the processing events, enhancing the proteolytic efficacy at all of the sites.

A general strategy for the development of antiviral agents is to inactivate virally encoded enzymes, including NS3, that are essential for the replication of the virus. Current efforts directed toward the discovery of NS3 protease inhibitors were reviewed by S. Tan, A. Pause, Y. Shi, N. Sonenberg, Hepatitis C Therapeutics: Current Status and Emerging Strategies, Nature Rev. Drug Discov., 1, 867-881 (2002). More relevant patent disclosures describing the synthesis of HCV protease inhibitors are: WO 2006/007700; US 2005/0261200; WO 2004/113365; WO 03/099274 (2003); US 2003/0008828; US2002/0037998 (2002); WO 00/59929 (2000); WO 00/09543 (2000); WO 99/50230 (1999); U.S. Pat. No. 5,861,297 (1999); WO 99/07733 (1999); US0267018 (2005); WO 06/043145 (2006); WO 06/086381 (2006); WO 07/025,307 (2007); WO 06/020276 (2006); WO 07/015,824 (2007); WO 07/016,441 (2007); WO 07/015,855 (2007); WO 07/015,787 (2007); WO 07/014,927 (2007); WO 07/014,926 (2007); WO 07/014,925 (2007); WO 07/014,924 (2007); WO 07/014,923 (2007); WO 07/014,922 (2007); WO 07/014,921 (2007); WO 07/014,920 (2007); WO 07/014,919 (2007); WO 07/014,918 (2007); WO 07/009,227 (2007); WO 07/008,657 (2007); WO 07/001,406 (2007); WO 07/011,658 (2007); WO 07/009,109 (2007); WO 06/119061 (2006).

### SUMMARY OF THE INVENTION

The present invention relates to novel HCV compounds and methods of treating a hepatitis C infection in a subject in need of such therapy with said HCV compounds. The present invention further relates to pharmaceutical compositions comprising the compounds of the present invention, or pharmaceutically acceptable salts, esters, or prodrugs thereof, in combination with a pharmaceutically acceptable carrier or excipient.

In one embodiment of the present invention, there are disclosed compounds of formulae I:

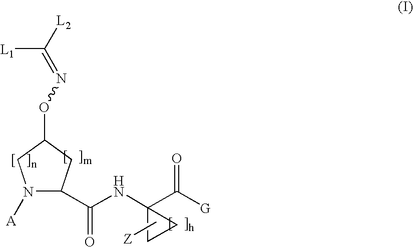

as well as the pharmaceutically acceptable salts, esters and prodrugs thereof, wherein:
A is selected from the group consisting of:
(1) R₁;
(2) (CO)R₁;
(3) (CO)OR₁;
(4) (CO)NR₁R₂;
(5) SO₂R₁;
(6) (SO₂)OR₁;
(7) SO₂NR₁R₂;
(8) (C═NR₁)NR₂R₃;
(9) (PO)R₁R₂;
(10) (PO)OR₁OR₂;
(11) (PO)NRINR₂;
(12) (PO)NR₁OR₂
R₁ and R₂ are independently selected from the group consisting of:
   a) hydrogen;
   b) aryl;
   c) substituted aryl;
   d) heteroaryl;
   e) substituted heteroaryl;
   f) heterocyclic or substituted heterocyclic;
   g) —C₁-C₈ alkyl, —C₂-C₈ alkenyl, or —C₂-C₈ alkynyl containing 0, 1, 2, or 3 heteroatoms selected from O, S or N;
   h) substituted —C₁-C₈ alkyl, substituted —C₂-C₈ alkenyl, or substituted —C₂-C₈ alkynyl containing 0, 1, 2, or 3 heteroatoms selected from O, S or N;
   i) —C₃-C₁₂ cycloalkyl;
   j) substituted —C₃-C₁₂ cycloalkyl;
   k) —C₃-C₁₂ cycloalkenyl;
   l) substituted —C₃-C₁₂ cycloalkenyl;
      or R₁ and R₂ taken together with the nitrogen atom to which they are attached form a substituted or unsubstituted heterocyclic;
      L₁ and L₂ are independently selected from the group consisting of:
(1) hydrogen;
(2) aryl;
(3) substituted aryl;
(4) heteroaryl;
(5) substituted heteroaryl;
(6) heterocyclic or substituted heterocyclic;
(7) —C₁-C₈ alkyl, —C₂-C₈ alkenyl, or —C₂-C₈ alkynyl containing 0, 1, 2, or 3 heteroatoms selected from O, S or N;
(8) substituted —C₁-C₈ alkyl, substituted —C₂-C₈ alkenyl, or substituted —C₂-C₈ alkynyl containing 0, 1, 2, or 3 heteroatoms selected from O, S or N;
(9) —C₃-C₁₂ cycloalkyl;
(10) substituted —C₃-C₁₂ cycloalkyl;
(11) —C₃-C₁₂ cycloalkenyl;
(12) substituted —C₃-C₁₂ cycloalkenyl;
(13)-Q-R₄, where Q is (CO), (CO)O, (CO)NR₅, (SO), (SO₂), (SO₂)NR₅; and R₄ and R₅ are independently selected from the group consisting of:
   a) hydrogen;
   b) aryl;
   c) substituted aryl;
   d) heteroaryl;
   e) substituted heteroaryl;
   f) heterocyclic or substituted heterocyclic;
   g) —C₁-C₈ alkyl, —C₂-C₈ alkenyl, or —C₂-C₈ alkynyl containing 0, 1, 2, or 3 heteroatoms selected from O, S or N;
   h) substituted —C₁-C₈ alkyl, substituted —C₂-C₈ alkenyl, or substituted —C₂-C₈ alkynyl containing 0, 1, 2, or 3 heteroatoms selected from O, S or N;
   i) —C₃-C₁₂ cycloalkyl;
   j) substituted —C₃-C₁₂ cycloalkyl;
   k) —C₃-C₁₂ cycloalkenyl;
   l) substituted —C₃-C₁₂ cycloalkenyl;
      or L₁ and L₂ taken together with the carbon atom to which they are attached form a cyclic moiety selected from: substituted or unsubstituted cycloalkyl, cycloalkenyl, or heterocylic; substituted or unsubstituted cycloalkyl, cycloalkenyl, and heterocyclic fused with one or more R₄; where R₄ is as previously defined;
      G is -E-R₄; and where E is absent, or E is O, CO, (CO)O, (CO)NR₅, NH, NH(CO), NH(CO)NR₅, NH(SO₂)NR₅ or NHSO₂; where R₄ and R₅ are as previously defined; or R₄ and R₅ taken together with the nitrogen atom to which they are attached form a substituted or unsubstituted heterocylic;
      Z is independently selected from the group consisting of:
(1) hydrogen;
(2) aryl;
(3) substituted aryl;
(4) heteroaryl;
(5) substituted heteroaryl;
(6) heterocyclic or substituted heterocyclic;
(7) —C₁-C₈ alkyl, —C₂-C₈ alkenyl, or —C₂-C₈ alkynyl containing 0, 1, 2, or 3 heteroatoms selected from O, S or N;
(8) substituted —C₁-C₈ alkyl, substituted —C₂-C₈ alkenyl, or substituted —C₂-C₈ alkynyl containing 0, 1, 2, or 3 heteroatoms selected from O, S or N;
(9) —C₃-C₁₂ cycloalkyl;
(10) substituted —C₃-C₁₂ cycloalkyl;
(11) —C₃-C₁₂ cycloalkenyl;
(12) substituted —C₃-C₁₂ cycloalkenyl;
   h=0, 1, 2 or 3;
   m=0, 1, 2 or 3;
   n=1, 2 or 3.

In another embodiment of the present invention there are disclosed pharmaceutical compositions comprising a therapeutically effective amount of a compound of the invention in combination with a pharmaceutically acceptable carrier or excipient. In yet another embodiment of the invention are methods of treating a hepatitis C infection in a subject in need of such treatment with said pharmaceutical compositions.

### DETAILED DESCRIPTION OF THE INVENTION

In a first embodiment of the present invention is a compound of formulae I as illustrated above, or a pharmaceutically acceptable salt, ester or prodrug thereof.

In a second embodiment of the present invention relates to compound of formulae II, or a pharmaceutically acceptable salt, ester or prodrug thereof:

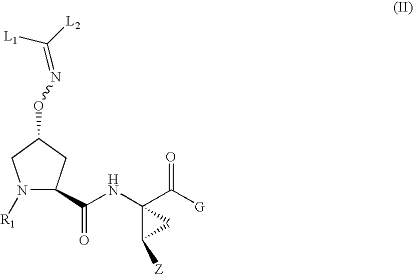

where R₁, G, L₁, L₂ and Z are as previously defined.

In a third embodiment of the present invention relates to compound of formulae III, or a pharmaceutically acceptable salt, ester or prodrug thereof:

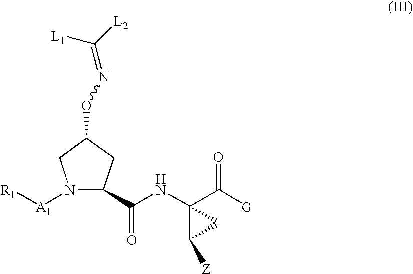

where A₁ is selected from —CO—, —SO₂—; where R₁, G, L₁, L₂ and Z are as previously defined. In a preferred embodiment, R₁ is not hydrogen. Preferably, R₁ is selected from:
(1) hydrogen;
(2) selected from, but not limited to structures (1)-(10):

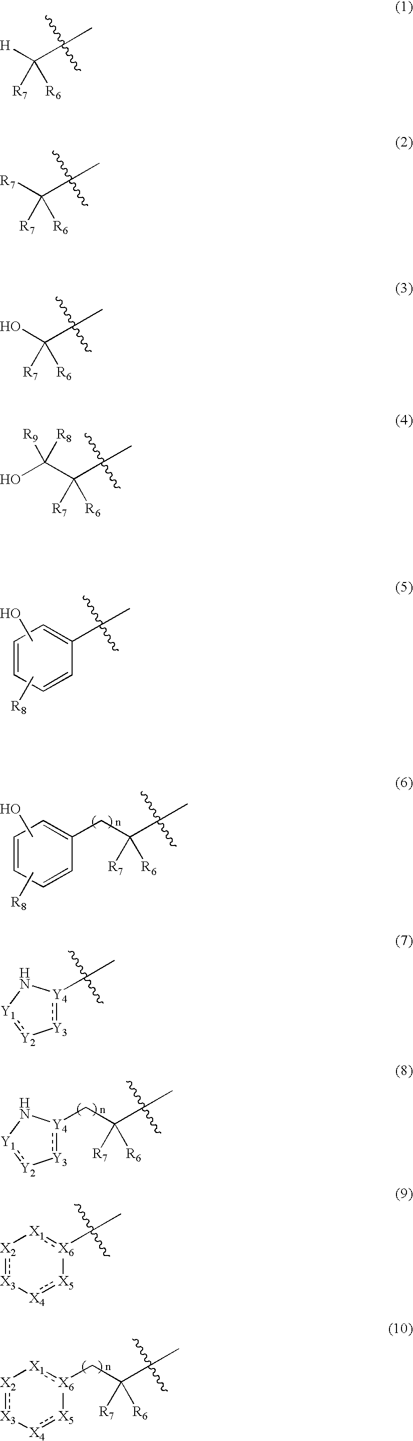

where R₆, R₇, R₈, R₉ are independently selected from the group consisting of:
a) hydrogen;
b) aryl;
c) substituted aryl;
d) heteroaryl;
e) substituted heteroaryl;
f) heterocyclic or substituted heterocyclic;
g) —C₁-C₈ alkyl, —C₂-C₈ alkenyl, or —C₂-C₈ alkynyl containing 0, 1, 2, or 3 heteroatoms selected from O, S or N;
h) substituted —C₁-C₈ alkyl, substituted —C₂-C₈ alkenyl, or substituted —C₂-C₈ alkynyl containing 0, 1, 2, or 3 heteroatoms selected from O, S or N;
i) —C₃-C₁₂ cycloalkyl;
j) substituted —C₃-C₁₂ cycloalkyl;
k) —C₃-C₁₂ cycloalkenyl;
l) substituted —C₃-C₁₂ cycloalkenyl;
   or R₆ and R₇ taken together with the carbon atom to which they are attached form a cyclic moiety;
   where X₁-X₅ are independently selected from —CO—, —CH—, —NH—, —O— and —N—; there's at least one —NH— among X₁-X₅; X₆ is selected from —C—, —CH—, —N—; X₁-X₅ can be further substituted when it is a CH or NH;
   Where Y₁-Y₃ are independently selected from CO, CH, NH, N, S and O; and Y₁-Y₃ can be further substituted when it is CH or NH; Y₄ is selected from C, CH and N; n=0, 1, 2;

In a fourth embodiment of the present invention relates to compound of formulae IV, or a pharmaceutically acceptable salt, ester or prodrug thereof:

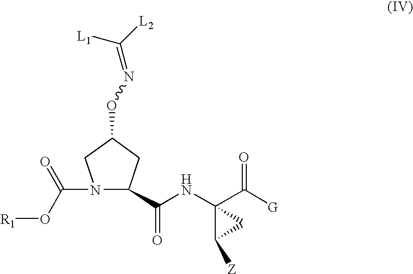

where R₁, G, L₁, L₂ and Z are as previously defined.

In a fifth embodiment of the present invention relates to compound of formulae V, or a pharmaceutically acceptable salt, ester or prodrug thereof:

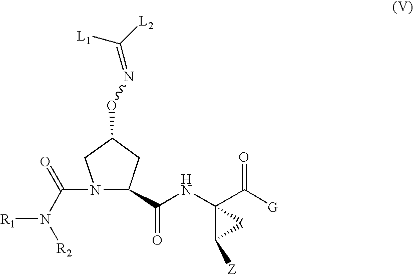

where R₁, R₂, G, L₁, L₂ and Z are as previously defined.

In a sixth embodiment of the present invention relates to compound of formulae VI, or a pharmaceutically acceptable salt, ester or prodrug thereof:

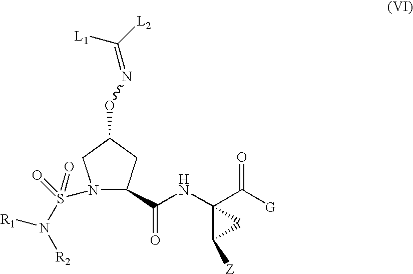

where R₁, R₂, G, L₁, L₂ and Z are as previously defined.

In a seventh embodiment of the present invention relates to compound of formulae VII, or a pharmaceutically acceptable salt, ester or prodrug thereof:

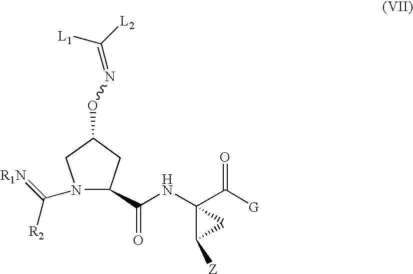

where R₁, R₂, G, L₁, L₂ and Z are as previously defined.

In a eighth embodiment of the present invention relates to compound of formulae VIII, or a pharmaceutically acceptable salt, ester or prodrug thereof:

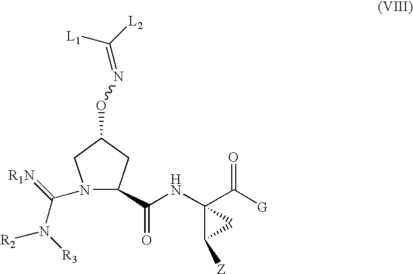

where R₁, R₂, R₃, G, L₁, L₂ and Z are as previously defined.

In a ninth embodiment of the present invention relates to compound of formulae IX, or a pharmaceutically acceptable salt, ester or prodrug thereof:

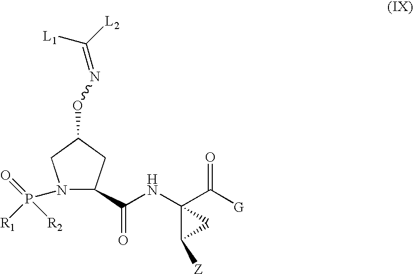

where R₁, R₂, G, L₁, L₂ and Z are as previously defined.

In a tenth embodiment of the present invention relates to compound of formulae X, or a pharmaceutically acceptable salt, ester or prodrug thereof:

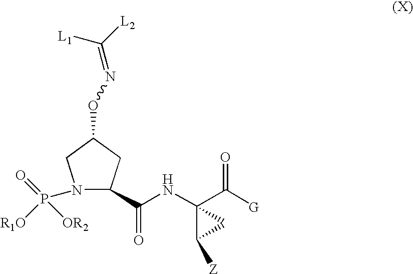

where R₁, R₂, G, L₁, L₂ and Z are as previously defined.

In a eleventh embodiment of the present invention relates to compound of formulae XI, or a pharmaceutically acceptable salt, ester or prodrug thereof:

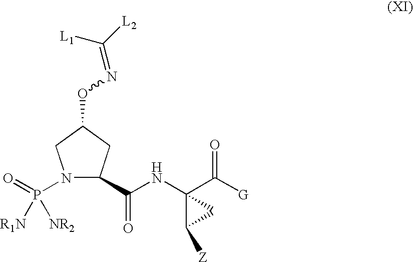

where R₁, R₂, G, L₁, L₂ and Z are as previously defined.

Representative compounds according to the invention include, but not limited to those examples (1)-(160) of the formula XII in Table 1:

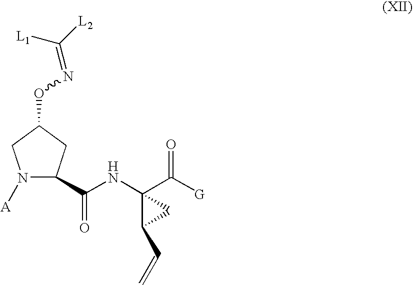

L₁, L₂, A and G are delineated for each example in TABLE 1:

TABLE 1

| Example | A | L₁L₂ | G |
|---|---|---|---|
| 1 | 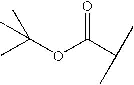 | 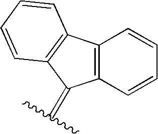 | 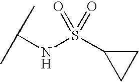 |
| 2 | 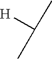 | 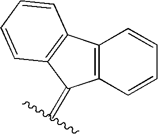 | 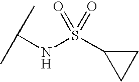 |
| 3 | 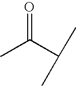 | 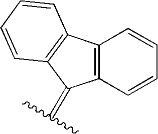 | 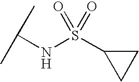 |
| 4 | 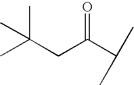 | 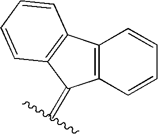 | 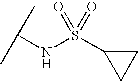 |
| 5 | 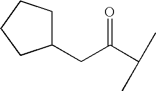 | 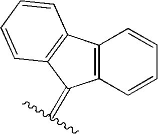 | 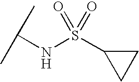 |
| 6 | 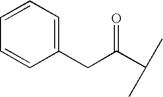 | 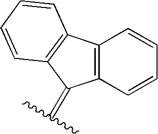 | 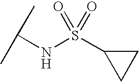 |
| 7 | 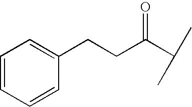 | 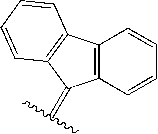 | 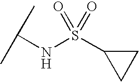 |
| 8 | 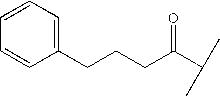 | 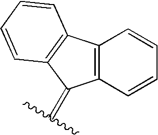 | 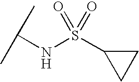 |
| 9 | 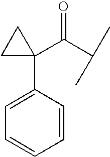 | 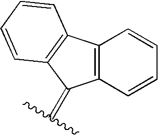 | 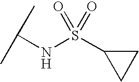 |
| 10 | 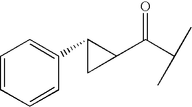 | 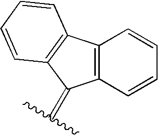 | 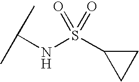 |
| 11 | 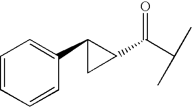 | 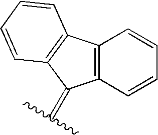 | 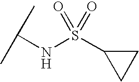 |
| 12 | 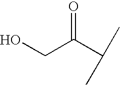 | 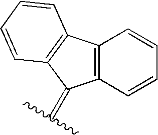 | 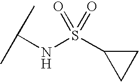 |
| 13 | 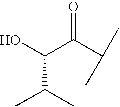 | 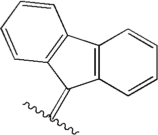 | 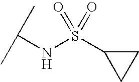 |
| 14 | 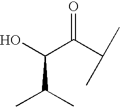 | 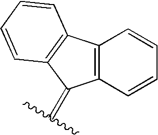 | 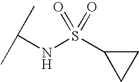 |
| 15 | 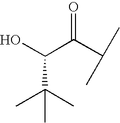 | 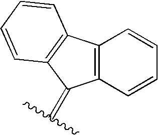 | 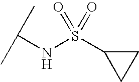 |
| 16 | 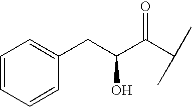 | 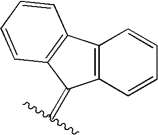 | 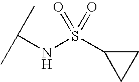 |
| 17 | 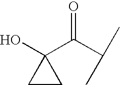 | 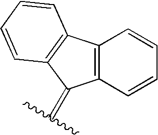 | 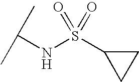 |
| 18 | 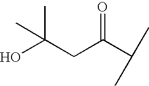 | 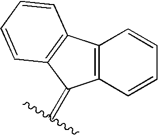 | 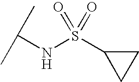 |
| 19 | 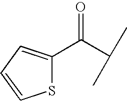 | 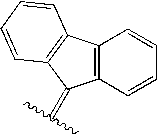 | 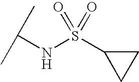 |
| 20 | 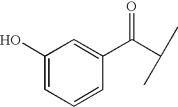 | 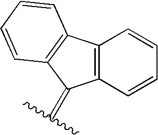 | 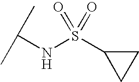 |
| 21 | 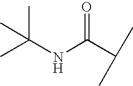 | 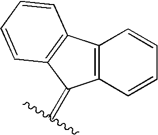 | 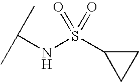 |
| 22 | 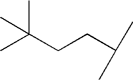 | 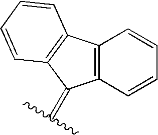 | 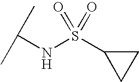 |
| 23 | 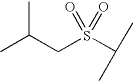 | 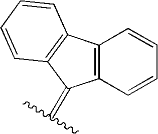 | 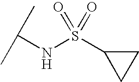 |
| 24 | 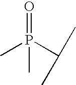 | 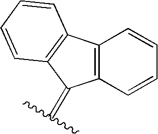 | 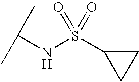 |
| 25 | 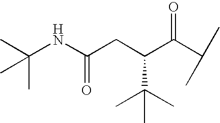 | 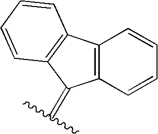 | 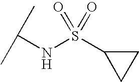 |
| 26 | 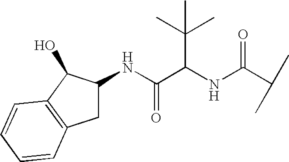 | 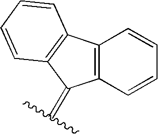 | 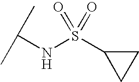 |
| 27 | 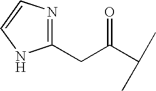 | 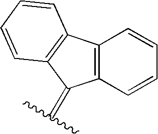 | 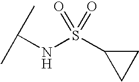 |
| 28 | 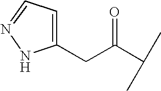 | 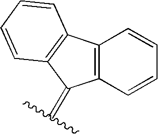 | 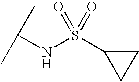 |
| 29 | 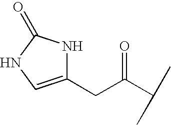 | 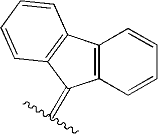 | 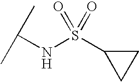 |
| 30 | 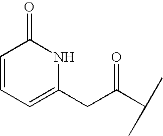 | 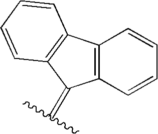 | 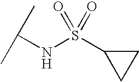 |
| 31 | 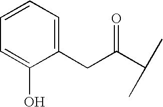 | 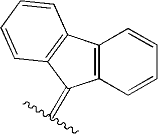 | 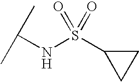 |
| 32 | 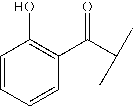 | 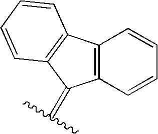 | 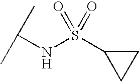 |
| 33 | 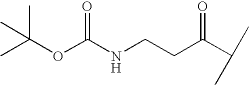 | 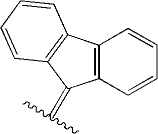 | 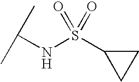 |
| 34 | 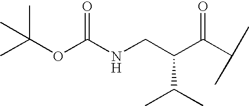 | 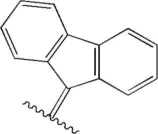 | 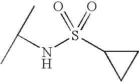 |
| 35 | 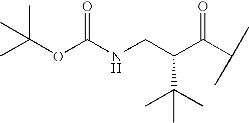 | 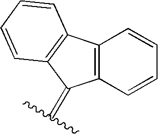 | 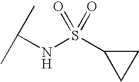 |
| 36 | 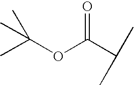 | 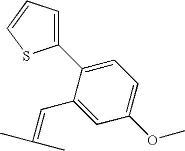 | 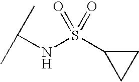 |
| 37 | 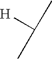 | 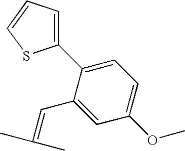 | 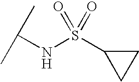 |
| 38 | 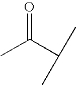 | 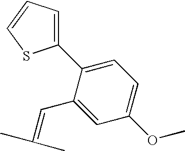 | 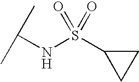 |
| 39 | 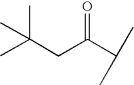 | 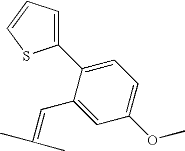 | 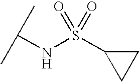 |
| 40 | 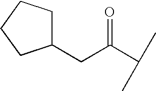 | 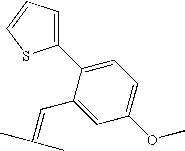 | 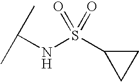 |
| 41 | 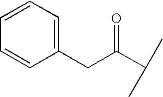 | 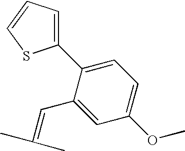 | 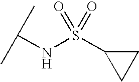 |
| 42 | 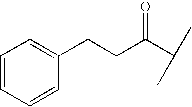 | 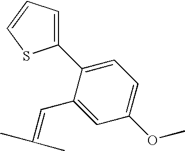 | 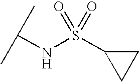 |
| 43 | 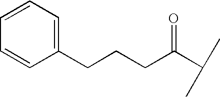 | 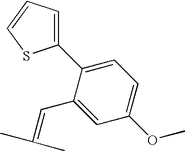 | 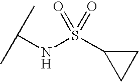 |
| 44 | 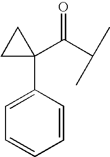 | 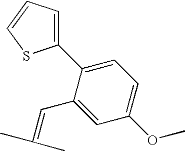 | 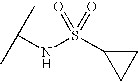 |
| 45 | 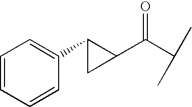 | 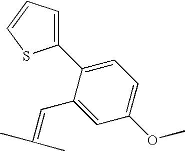 | 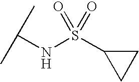 |
| 46 | 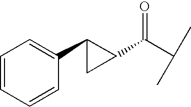 | 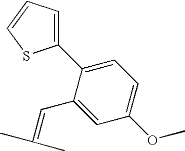 | 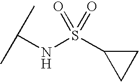 |
| 47 | 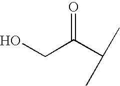 | 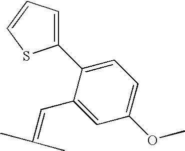 | 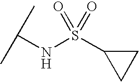 |
| 48 | 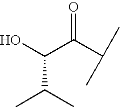 | 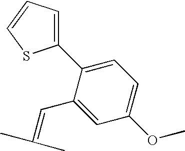 | 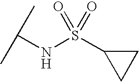 |
| 49 | 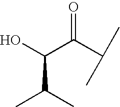 | 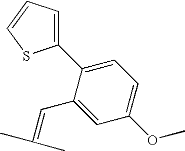 | 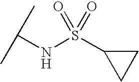 |
| 50 | 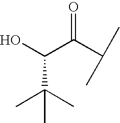 | 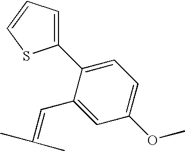 | 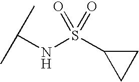 |
| 51 | 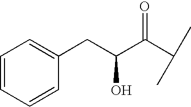 | 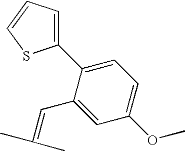 | 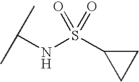 |
| 52 | 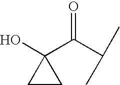 | 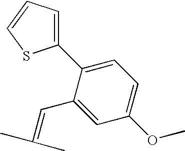 | 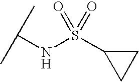 |
| 53 | 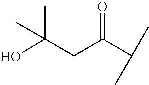 | 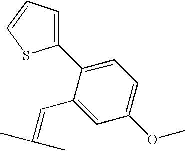 | 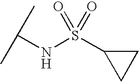 |
| 54 | 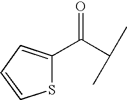 | 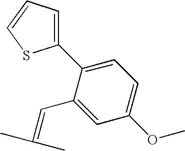 | 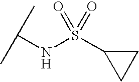 |
| 55 | 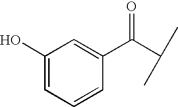 | 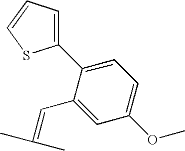 | 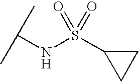 |
| 56 | 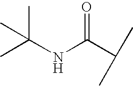 | 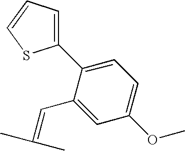 | 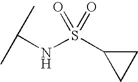 |
| 57 | 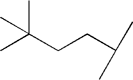 | 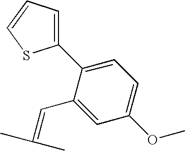 | 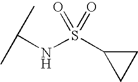 |
| 58 | 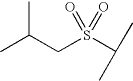 | 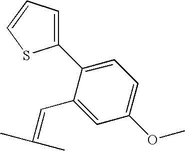 | 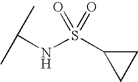 |
| 59 | 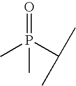 | 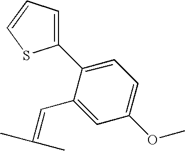 | 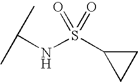 |
| 60 | 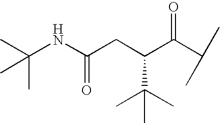 | 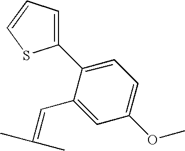 | 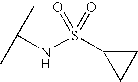 |
| 61 | 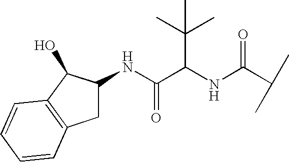 | 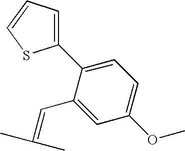 | 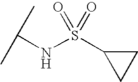 |
| 62 | 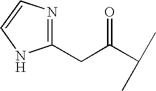 | 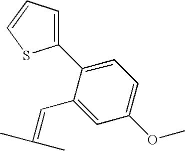 | 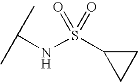 |
| 63 | 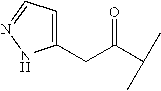 | 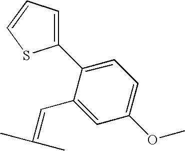 | 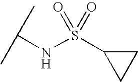 |
| 64 | 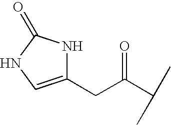 | 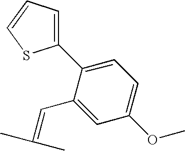 | 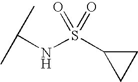 |
| 65 | 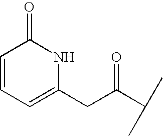 | 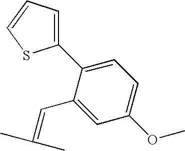 | 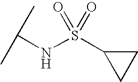 |
| 66 | 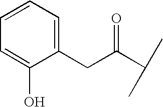 | 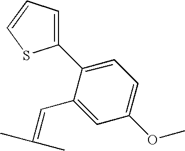 | 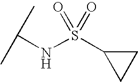 |
| 67 | 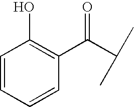 | 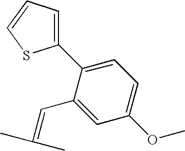 | 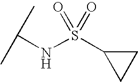 |
| 68 | 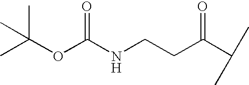 | 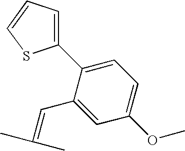 | 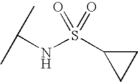 |
| 69 | 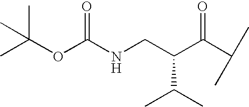 | 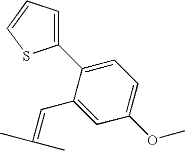 | 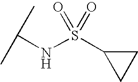 |
| 70 | 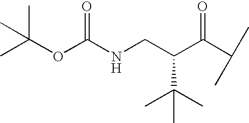 | 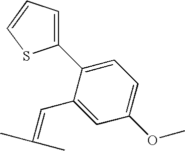 | 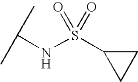 |
| 71 | 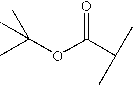 | 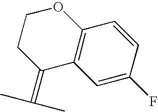 | 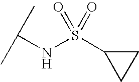 |
| 72 | 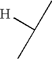 | 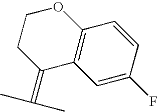 | 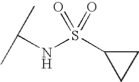 |
| 73 | 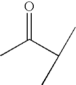 | 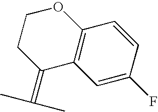 | 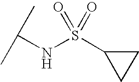 |
| 74 | 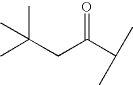 | 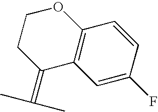 | 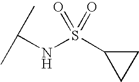 |
| 75 | 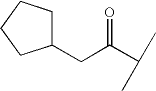 | 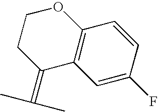 | 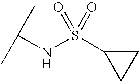 |
| 76 | 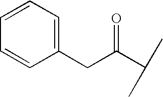 | 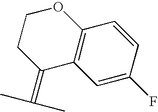 | 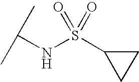 |
| 77 | 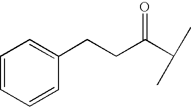 | 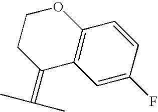 | 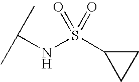 |
| 78 | 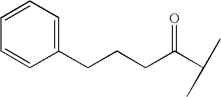 | 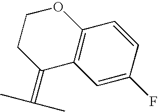 | 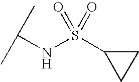 |
| 79 | 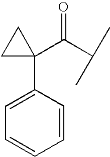 | 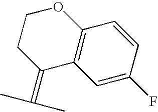 | 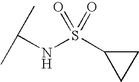 |
| 80 | 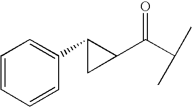 | 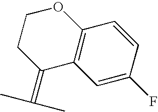 | 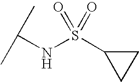 |
| 81 | 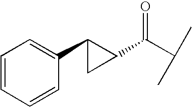 | 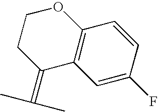 | 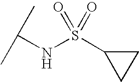 |
| 82 | 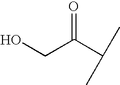 | 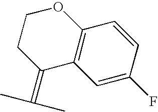 | 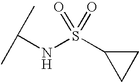 |
| 83 | 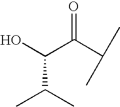 | 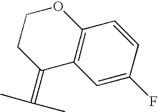 | 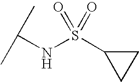 |
| 84 | 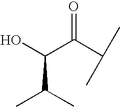 | 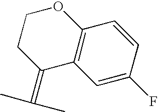 | 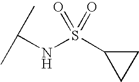 |
| 85 | 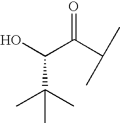 | 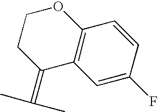 | 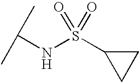 |
| 86 | 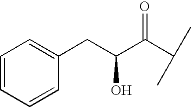 | 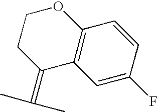 | 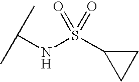 |
| 87 | 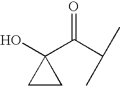 | 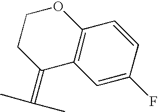 | 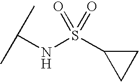 |
| 88 | 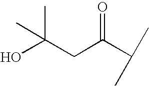 | 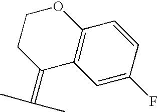 | 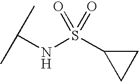 |
| 89 | 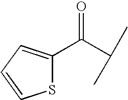 | 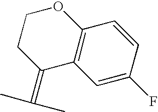 | 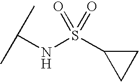 |
| 90 | 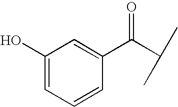 | 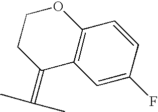 | 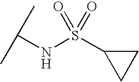 |
| 91 | 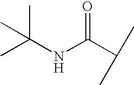 | 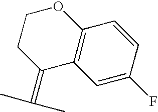 | 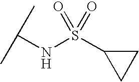 |
| 92 | 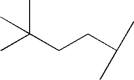 | 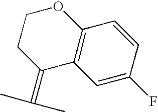 | 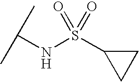 |
| 93 | 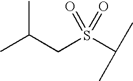 | 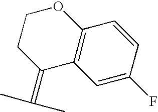 | 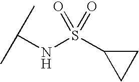 |
| 94 | 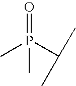 | 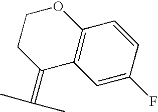 | 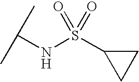 |
| 95 | 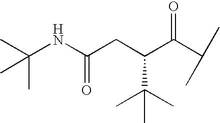 | 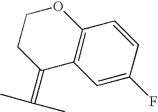 | 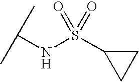 |
| 96 | 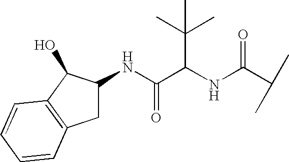 | 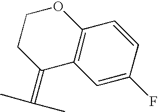 | 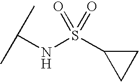 |
| 97 | 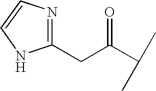 | 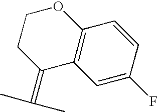 | 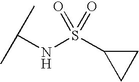 |
| 98 | 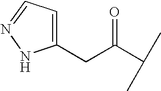 | 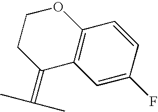 | 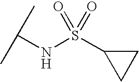 |
| 99 | 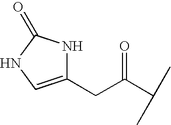 | 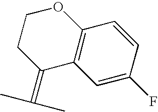 | 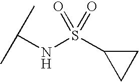 |
| 100 | 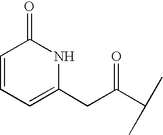 | 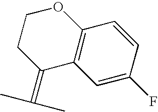 | 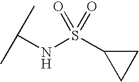 |
| 101 | 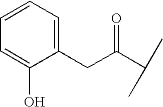 | 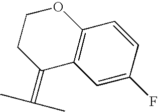 | 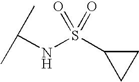 |
| 102 | 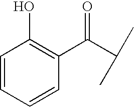 | 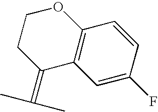 | 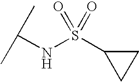 |
| 103 | 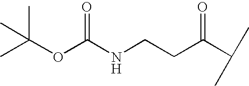 | 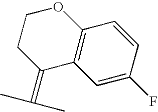 | 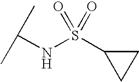 |
| 104 | 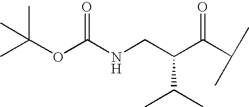 | 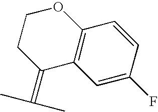 | 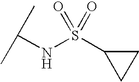 |
| 105 | 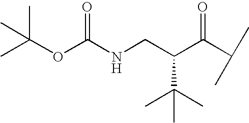 | 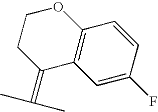 | 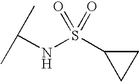 |
| 106 | 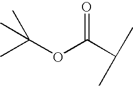 | 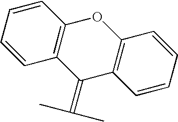 | 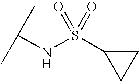 |
| 107 | 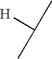 | 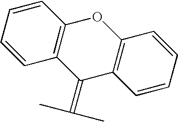 | 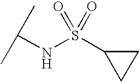 |
| 108 | 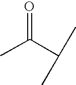 | 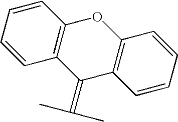 | 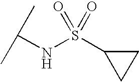 |
| 109 | 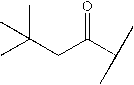 | 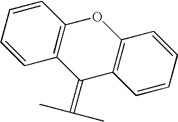 | 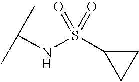 |
| 110 | 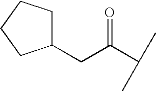 | 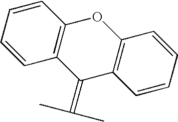 | 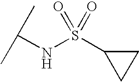 |
| 111 | 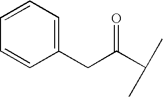 | 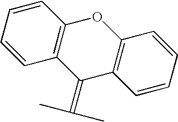 | 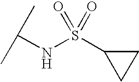 |
| 112 | 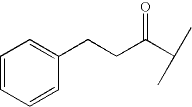 | 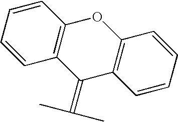 | 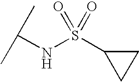 |
| 113 | 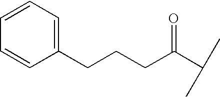 | 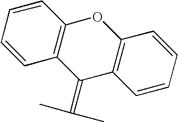 | 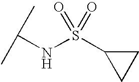 |
| 114 | 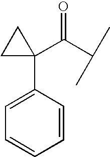 | 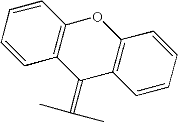 | 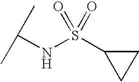 |
| 115 | 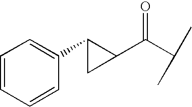 | 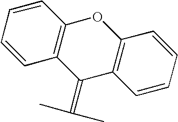 | 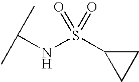 |
| 116 | 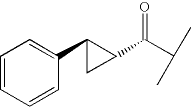 | 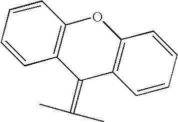 | 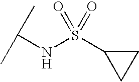 |
| 117 | 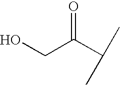 | 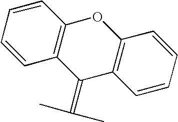 | 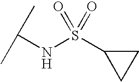 |
| 118 | 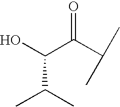 | 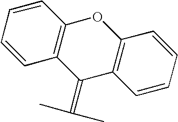 | 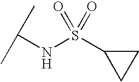 |
| 119 | 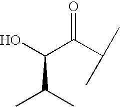 | 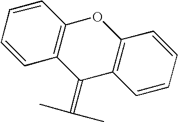 | 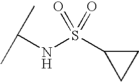 |
| 120 | 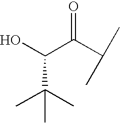 | 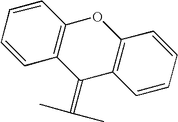 | 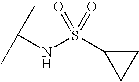 |
| 121 | 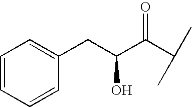 | 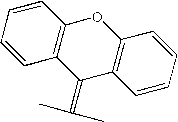 | 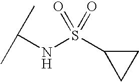 |
| 122 | 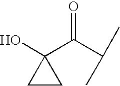 | 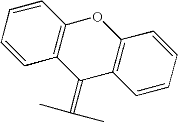 | 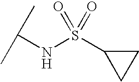 |
| 123 | 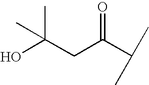 | 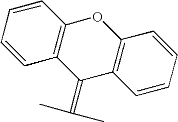 | 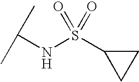 |
| 124 | 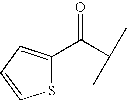 | 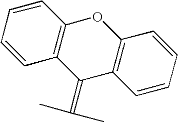 | 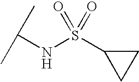 |
| 125 | 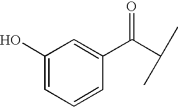 | 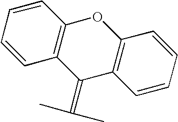 | 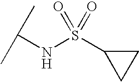 |
| 126 | 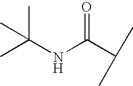 | 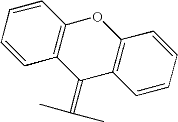 | 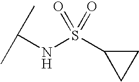 |
| 127 | 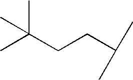 | 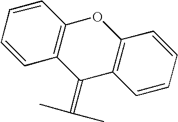 | 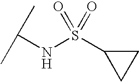 |
| 128 | 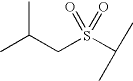 | 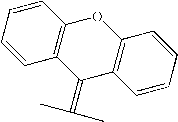 | 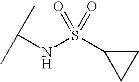 |
| 129 | 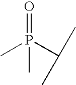 | 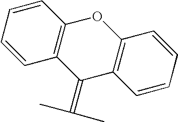 | 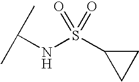 |
| 130 | 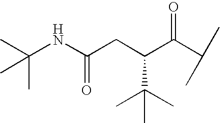 | 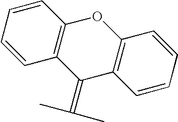 | 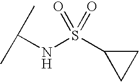 |
| 131 | 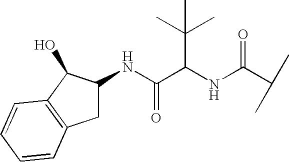 | 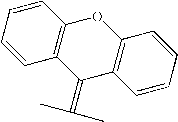 | 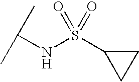 |
| 132 | 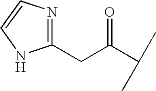 | 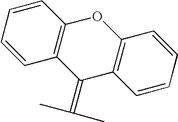 | 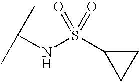 |
| 133 | 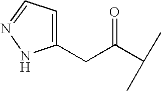 | 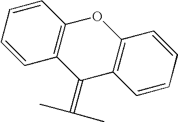 | 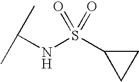 |
| 134 | 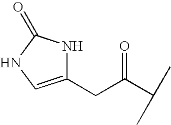 | 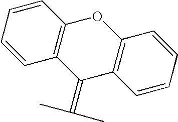 | 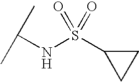 |
| 135 | 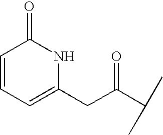 | 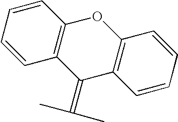 | 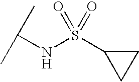 |
| 136 | 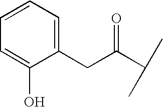 | 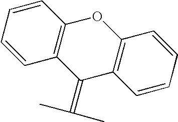 | 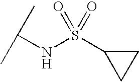 |
| 137 | 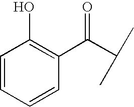 | 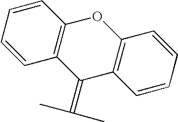 | 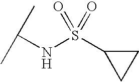 |
| 138 | 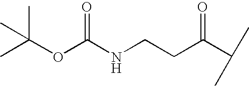 | 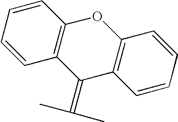 | 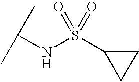 |
| 139 | 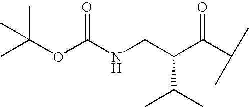 | 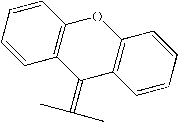 | 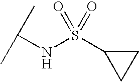 |
| 140 | 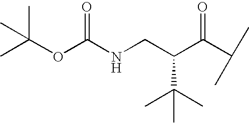 | 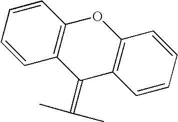 | 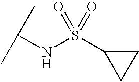 |
| 141 | 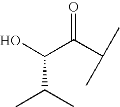 | 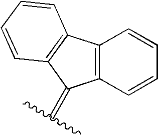 | 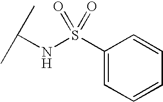 |
| 142 | 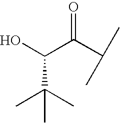 | 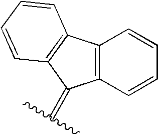 | 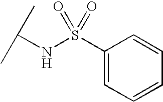 |
| 143 | 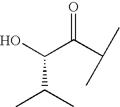 | 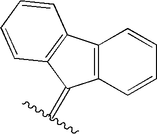 | 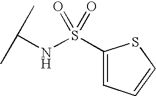 |
| 144 | 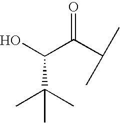 | 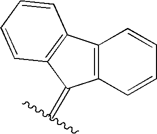 | 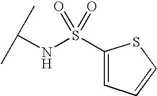 |
| 145 | 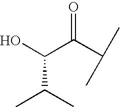 | 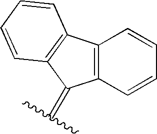 | 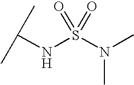 |
| 146 | 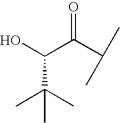 | 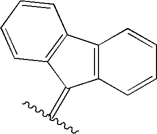 | 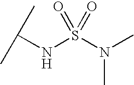 |
| 147 | 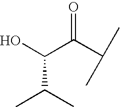 | 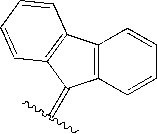 | 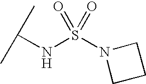 |
| 148 | 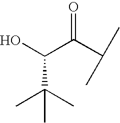 | 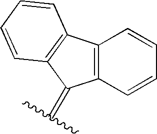 | 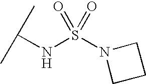 |
| 149 | 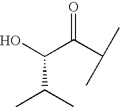 | 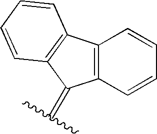 | 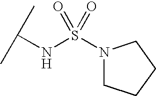 |
| 150 | 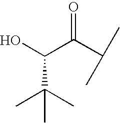 | 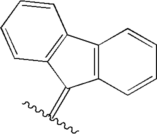 | 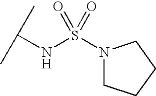 |
| 151 | 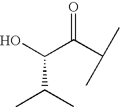 | 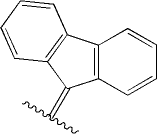 | 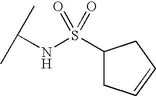 |
| 152 | 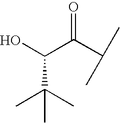 | 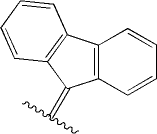 | 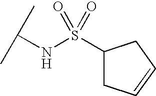 |
| 153 | 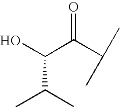 | 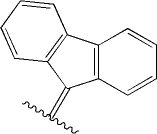 | 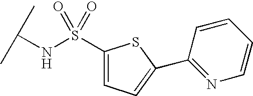 |
| 154 | 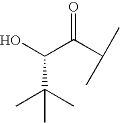 | 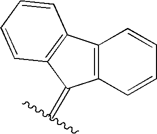 | 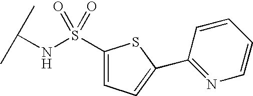 |
| 155 | 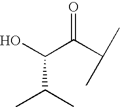 | 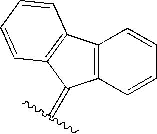 | 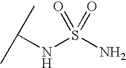 |
| 156 | 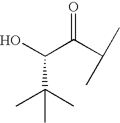 | 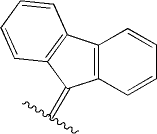 | 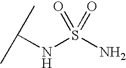 |
| 157 | 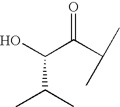 | 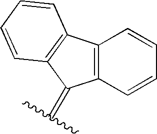 | 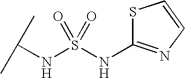 |
| 158 | 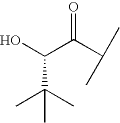 | 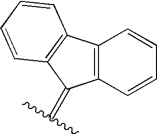 | 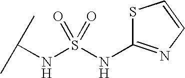 |
| 159 | 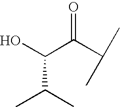 | 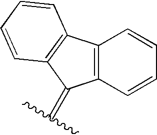 | 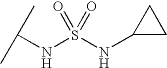 |
| 160 | 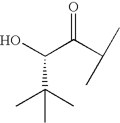 | 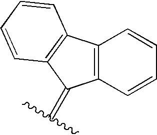 | 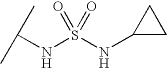 |

A further embodiment of the present invention includes pharmaceutical compositions comprising any single compound delineated herein, or a pharmaceutically acceptable salt, ester, or prodrug thereof, with a pharmaceutically acceptable carrier or excipient.

Yet another embodiment of the present invention is a pharmaceutical composition comprising a combination of two or more compounds delineated herein, or a pharmaceutically acceptable salt, ester, or prodrug thereof, with a pharmaceutically acceptable carrier or excipient.

Yet a further embodiment of the present invention is a pharmaceutical composition comprising any single compound delineated herein in combination with one or more HCV compounds known in the art, or a pharmaceutically acceptable salt, ester, or prodrug thereof, with a pharmaceutically acceptable carrier or excipient.

### DEFINITIONS

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system having one or two aromatic rings including, but not limited to, phenyl, naphthyl, tetrahydronaphthyl, indanyl, idenyl and the like.

The term “heteroaryl,” as used herein, refers to a mono- or polycyclic (e.g. bi-, or tri-cyclic or more), fused or non-fused, aromatic radical or ring having from five to ten ring atoms of which one or more ring atom is selected from, for example, S, O and N; zero, one or two ring atoms are additional heteroatoms independently selected from, for example, S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “C₁-C₈ alkyl,” or “C₁-C₁₂ alkyl,” as used herein, refer to saturated, straight- or branched-chain hydrocarbon radicals containing between one and eight, or one and twelve carbon atoms, respectively. Examples of C₁-C₈ alkyl radicals include, but are not limited to, methyl, ethyl, propyl, isopropyl, n-butyl, tert-butyl, neopentyl, n-hexyl, heptyl and octyl radicals; and examples of C₁-C₁₂ alkyl radicals include, but are not limited to, methyl, ethyl, propyl, isopropyl, n-butyl, tert-butyl, neopentyl, n-hexyl, heptyl, octyl, decyl, dodecyl radicals.

The term “C₂-C₈ alkenyl,” as used herein, denotes a monovalent group derived from a hydrocarbon moiety containing from two to eight carbon atoms having at least one carbon-carbon double bond by the removal of a single hydrogen atom. Alkenyl groups include, but are not limited to, for example, ethenyl, propenyl, butenyl, 1-methyl-2-buten-1-yl, heptenyl, octenyl and the like.

The term “C₂-C₈ alkynyl,” as used herein, denotes a monovalent group derived from a hydrocarbon moiety containing from two to eight carbon atoms having at least one carbon-carbon triple bond by the removal of a single hydrogen atom. Representative alkynyl groups include, but are not limited to, for example, ethynyl, 1-propynyl, 1-butynyl, heptynyl, octynyl and the like.

The term “C₃-C₈-cycloalkyl”, or “C₃-C₁₂-cycloalkyl,” as used herein, denotes a monovalent group derived from a monocyclic or polycyclic saturated carbocyclic ring compound by the removal of a single hydrogen atom, respectively. Examples of C₃-C₈-cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo[2.2.1]heptyl, and bicyclo[2.2.2]octyl.

The term “C₃-C₈-cycloalkenyl”, or “C₃-C₁₂-cycloalkenyl” as used herein, denote a monovalent group derived from a monocyclic or polycyclic carbocyclic ring compound having at least one carbon-carbon double bond by the removal of a single hydrogen atom. Examples of C₃-C₈-cycloalkenyl include, but not limited to, cyclopropenyl, cyclobutenyl, cyclopentenyl, cyclohexenyl, cycloheptenyl, cyclooctenyl, and the like; and examples of C₃-C₁₂-cycloalkenyl include, but not limited to, cyclopropenyl, cyclobutenyl, cyclopentenyl, cyclohexenyl, cycloheptenyl, cyclooctenyl, and the like.

The terms “substituted” “substituted aryl”, “substituted heteroaryl,” “substituted C₁-C₈ alkyl,” “substituted C₂-C₈ alkenyl,” “substituted C₂-C₈ alkynyl”, “substituted C₃-C₈ cycloalkyl,” “substituted C₃-C₁₂ cycloalkyl,” “substituted C₃-C₈ cycloalkenyl,” “substituted C₃-C₁₂ cycloalkenyl,” as used herein, refer to CH, NH, aryl, heteroaryl, C₁-C₈ alkyl, C₂-C₈ alkenyl, C₂-C₈ alkynyl, C₃-C₈ cycloalkyl, C₃-C₁₂ cycloalkyl, C₃-C₈ cycloalkenyl, C₃-C₁₂ cycloalkenyl groups as previously defined, substituted by independent replacement of one, two, or three or more of the hydrogen atoms thereon with substituents including, but not limited to, —F, —Cl, —Br, —I, —OH, protected hydroxy, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkenyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₁₂-alkenyl, —OCO₂—C₂-C₁₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkenyl, —OCONH—C₃-C₁₂-cycloalkyl, —OCONH— aryl, —OCONH— heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂— aryl, —NHCO₂— heteroaryl, —NHCO₂-heterocycloalkyl, —NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC H)— aryl, —NHC(NH)— heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkenyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)—aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH— aryl, —SO₂NH-heteroaryl, —SO₂NH— heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S-heterocycloalkyl, or methylthiomethyl. It is understood that the aryls, heteroaryls, alkyls, and the like can be further substituted.

In accordance with the invention, any of the aryls, substituted aryls, heteroaryls and substituted heteroaryls described herein, can be any aromatic group. Aromatic groups can be substituted or unsubstituted.

It is understood that any alkyl, alkenyl, alkynyl, cycloalkyl and cycloalkenyl moiety described herein can also be an aliphatic group, an alicyclic group or a heterocyclic group. An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may be used in place of the alkyl, alkenyl, alkynyl, alkylene, alkenylene, and alkynylene groups described herein.

The term “alicyclic,” as used herein, denotes a monovalent group derived from a monocyclic or polycyclic saturated carbocyclic ring compound by the removal of a single hydrogen atom. Examples include, but not limited to, cyclopropyl, cyclobutyl, cyclopentyl, cyclohexyl, bicyclo[2.2.1]heptyl, and bicyclo[2.2.2]octyl. Such alicyclic groups may be further substituted.

The term “heterocyclic” as used herein, refers to a non-aromatic 5-, 6- or 7-membered ring or a bi- or tri-cyclic group fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl. Such heterocyclic groups may be further substituted to give substituted heterocyclic.

The term “halogen,” as used herein, refers to an atom selected from fluorine, chlorine, bromine and iodine.

The term “hydroxy activating group”, as used herein, refers to a labile chemical moiety which is known in the art to activate a hydroxy group so that it will depart during synthetic procedures such as in a substitution or an elimination reactions. Examples of hydroxy activating group include, but not limited to, mesylate, tosylate, triflate, p-nitrobenzoate, phosphonate and the like.

The term “activated hydroxy”, as used herein, refers to a hydroxy group activated with a hydroxy activating group, as defined above, including mesylate, tosylate, triflate, p-nitrobenzoate, phosphonate groups, for example.

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy protecting group,” as used herein, refers to a labile chemical moiety which is known in the art to protect a hydroxy group against undesired reactions during synthetic procedures. After said synthetic procedure(s) the hydroxy protecting group as described herein may be selectively removed. Hydroxy protecting groups as known in the are described generally in T. H. Greene and P. G. M. Wuts, Protective Groups in Organic Synthesis, 3rd edition, John Wiley & Sons, New York (1999). Examples of hydroxy protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl(trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxy protecting groups for the present invention are acetyl (Ac or —C(O)CH₃), benzoyl (Bz or —C(O)C₆H₅), and trimethylsilyl (TMS or —Si(CH₃)₃).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “protected amino,” as used herein, refers to an amino group protected with an amino protecting group as defined above.

The term “alkylamino” refers to a group having the structure —NH(C₁-C₁₂ alkyl) where C₁-C₁₂ alkyl is as previously defined.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates. Examples of aliphatic carbonyls include, but are not limited to, acetyl, propionyl, 2-fluoroacetyl, butyryl, 2-hydroxy acetyl, and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protogenic organic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As used herein, the term “substantially pure” for a compound refers to the physical state of said compound after being obtained from a purification process or processes described herein or that are well known to the skilled artisan, in sufficient purity to be characterizable by standard analytical techniques described herein or as are well known to the skilled artisan.

In one embodiment, a substantially pure compound comprises a compound of greater than about 75% purity. This means that the compound does not contain more than about 25% of any other compound. In one embodiment, a substantially pure compound comprises a compound of greater than about 80% purity. This means that the compound does not contain more than about 20% of any other compound. In one embodiment, a substantially pure compound comprises a compound of greater than about 85% purity. This means that the compound does not contain more than about 15% of any other compound. In one embodiment, a substantially pure compound comprises a compound of greater than about 90% purity. This means that the compound does not contain more than about 10% of any other compound. In another embodiment, a substantially pure compound comprises a compound of greater than about 95% purity. This means that the compound does not contain more than about 5% of any other compound. In another embodiment, a substantially pure compound comprises greater than about 98% purity. This means that the compound does not contain more than about 2% of any other compound. In one embodiment, a substantially pure compound comprises a compound of greater than about 99% purity. This means that the compound does not contain more than about 1% of any other compound.

As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of Formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

This invention also encompasses pharmaceutical compositions containing, and methods of treating bacterial infections through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxyysine, demo sine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intra-articular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, bacterial infections are treated or prevented in a patient such as a human or other animals by administering to the patient a therapeutically effective amount of a compound of the invention, in such amounts and for such time as is necessary to achieve the desired result.

By a “therapeutically effective amount” of a compound of the invention is meant a sufficient amount of the compound to treat or prevent bacterial infections, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

In yet another embodiment, the compounds of the invention may be used for the treatment of HCV in humans in monotherapy mode or in a combination therapy (e.g., dual combination, triple combination etc.) mode such as, for example, in combination with antiviral and/or immunomodulatory agents. Examples of such antiviral and/or immunomodulatory agents include Ribavirin (from Schering-Plough Corporation, Madison, N.J.) and Levovirin (from ICN Pharmaceuticals, Costa Mesa, Calif.), VP 50406 (from Viropharma, Incorporated, Exton, Pa.), ISIS 14803 (from ISIS Pharmaceuticals, Carlsbad, Calif.), Heptazyme™ (from Ribozyme Pharmaceuticals, Boulder, Colo.), VX 497, and Teleprevir (VX-950) (both from Vertex Pharmaceuticals, Cambridge, Mass.), Thymosin™ (from SciClone Pharmaceuticals, San Mateo, Calif.), Maxamine™ (Maxim Pharmaceuticals, San Diego, Calif.), mycophenolate mofetil (from Hoffman-LaRoche, Nutley, N.J.), interferon (such as, for example, interferon-alpha, PEG-interferon alpha conjugates) and the like. “PEG-interferon alpha conjugates” are interferon alpha molecules covalently attached to a PEG molecule. Illustrative PEG-interferon alpha conjugates include interferon alpha-2a (Roferon™, from Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™), interferon alpha-2c (BILB 1941, BILN 2061 and Berofor Alpha™, (all from Boehringer Ingelheim, Ingelheim, Germany), consensus interferon as defined by determination of a consensus sequence of naturally occurring interferon alphas (Infergen™, from Amgen, Thousand Oaks, Calif.). Other suitable anti-HCV agents for use in combination with the present invention include but are not limited to: Yeast-core-NS3 vaccine, Envelope Vaccine, A-837093 (Abbott Pharmaceuticals), AG0121541 (Pfizer), GS9132 (Gilead); HCV-796 (Viropharma), ITMN-191 (Intermune), JTK 003/109 (Japan Tobacco Inc.), Lamivudine (EPIVIR) (Glaxo Smith Kline), MK-608 (Merck), R803 (Rigel), ZADAXIN (SciClone Pharmaceuticals); Valopicitabine (Idenix), VGX-410C (Viralgenomix), R1626 (Hoffman La-Roche), and SCH-503034 (Schering Plough Corporation).

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

Unless otherwise defined, all technical and scientific terms used herein are accorded the meaning commonly known to one with ordinary skill in the art. All publications, patents, published patent applications, and other references mentioned herein are hereby incorporated by reference in their entirety.

### ABBREVIATIONS

Abbreviations which may appear in the following synthetic schemes and examples are:
Ac for acetyl;
Boc for tert-butoxycarbonyl;
Bz for benzoyl;
Bn for benzyl;
CDI for carbonyldiimidazole;
dba for dibenzylidene acetone;
DBU for 1,8-diazabicyclo[5.4.0]undec-7-ene;
DIAD for diisopropylazodicarboxylate;
DMAP for dimethylaminopyridine;
DMF for dimethyl formamide;
DMSO for dimethyl sulfoxide;
dppb for diphenylphosphino butane;
EtOAc for ethyl acetate;
HATU for 2-(7-Aza-1H-benzotriazole-1-yl)-1,1,3,3-tetramethyluronium hexafluorophosphate;
iPrOH for isopropanol;
NaHMDS for sodium bis(trimethylsilyl)amide;
NMO for N-methylmorpholine N-oxide;
MeOH for methanol;
Ph for phenyl;
POPd for dihydrogen dichlorobis(di-tert-butylphosphino)palladium(II);
TBAHS for tetrabutyl ammonium hydrogen sulfate;
TEA for triethylamine;
THF for tetrahydrofuran;
TPP for triphenylphosphine;
Tris for Tris(hydroxymethyl)aminomethane;
BME for 2-mercaptoethanol;
BOP for benzotriazol-1-yloxy-tris(dimethylamino)phosphonium hexafluorophosphate;
COD for cyclooctadiene;
DAST for diethylaminosulfur trifluoride;
DABCYL for 6-(N-4′-carboxy-4-(dimethylamino)azobenzene)-aminohexyl-1-O-(2-cyanoethyl)-(N,N-diisopropyl)-phosphoramidite;
DCM for dichloromethane;
DIBAL-H for diisobutylaluminum hydride;
DIEA for diisopropyl ethylamine;
DME for ethylene glycol dimethyl ether;
DMEM for Dulbecco's Modified Eagles Media;
EDANS for 5-(2-Amino-ethylamino)-naphthalene-1-sulfonic acid;
EDCI or EDC for 1-(3-diethylaminopropyl)-3-ethylcarbodiimide hydrochloride;
Hoveyda's Cat. for Dichloro(o-isopropoxyphenylmethylene) (tricyclohexylphosphine)ruthenium(II);
KHMDS is potassium bis(trimethylsilyl) amide;
Ms for mesyl;
NMM for N-4-methylmorpholine;
PyBrOP for Bromo-tri-pyrrolidino-phosphonium hexafluorophosphate;
RCM for ring-closing metathesis;
RT for reverse transcription;
RT-PCR for reverse transcription-polymerase chain reaction;
TEA for triethyl amine;
TFA for trifluoroacetic acid;
THF for tetrahydrofuran; and
TLC for thin layer chromatography.

### Synthetic Methods

The compounds and processes of the present invention will be better understood in connection with the following synthetic schemes that illustrate the methods by which the compounds of the invention may be prepared.

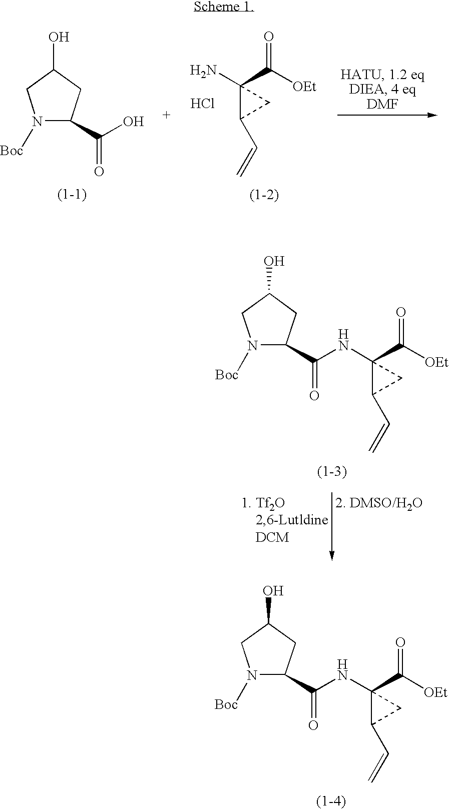

Scheme 1 describes the synthesis of intermediate (1-4). The trans dipeptide (1-3) was synthesized from Boc-trans-L-hydroxyproline (1-1) and (1R,2S)-Ethyl 1-amino-2-vinylcyclopropane carboxylate (1-2) via peptide coupling reaction with appropriate coupling reagent. The trans dipeptide (1-3) was converted to cis dipeptide through SN2 inversion of hydroxyl group by converting hydroxyl intermediate to a suitable leaving group such as, but not limited to OMs, OTs, OTf, bromide, or iodide.

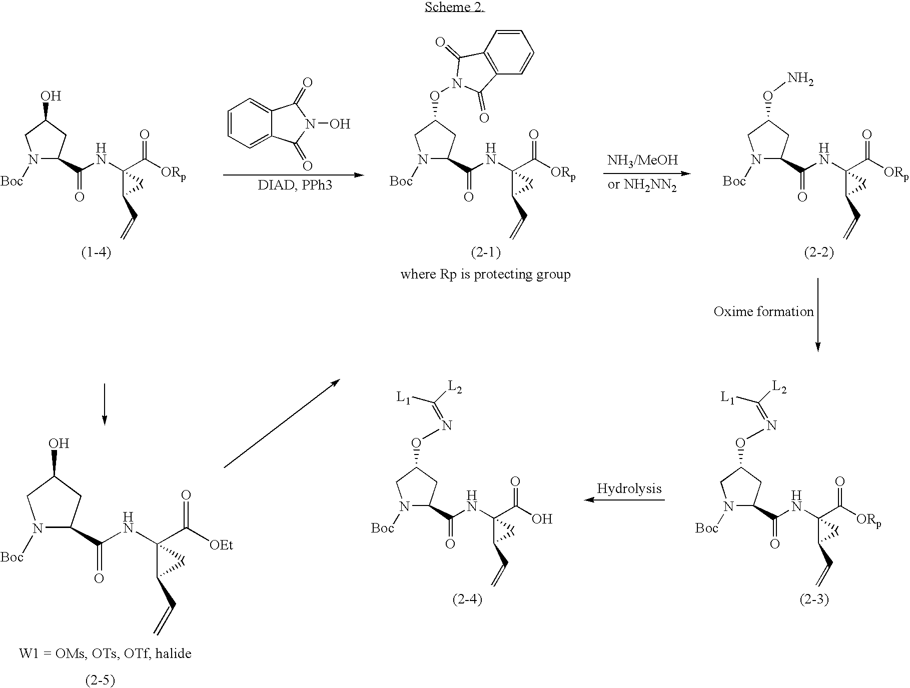

The analogs of the present invention were prepared via several different synthetic routes. The simplest method, shown in Scheme 2, is to condense commercially available hydroxyphthalimide using Mitsunobu conditions followed by deprotection of the phthalimide moiety with ammonia or hydrazine to provide hydroxy amine (2-2). For further details on the Mitsunobu reaction, see O. Mitsunobu, Synthesis 1981, 1-28; D. L. Hughes, Org. React. 29, 1-162 (1983); D. L. Hughes, Organic Preparations and Procedures Int. 28, 127-164 (1996); and J. A. Dodge, S. A. Jones, Recent Res. Dev. Org. Chem. 1, 273-283 (1997). Alternatively, intermediate (2-2) can also be made by converting hydroxy intermediate (1-4) to a suitable leaving group such as, but not limited to OMs, OTs, OTf, bromide, or iodide; followed with the deprotection of the phthalimide moiety with ammonia or hydrazine. Oximes (2-3) can be prepared by treating hydroxy amine with appropriate aldehyde or ketone optionally in the presence of an acid. Subsequent removal of the acid protecting group furnishes compounds of formula (2-4). A thorough discussion of solvents and conditions for protecting the acid group can be found in T. W Greene and P. G. M. Wuts, Protective Organic Synthesis, 3^{rd} ed., John Wiley & Son, Inc, 1999.

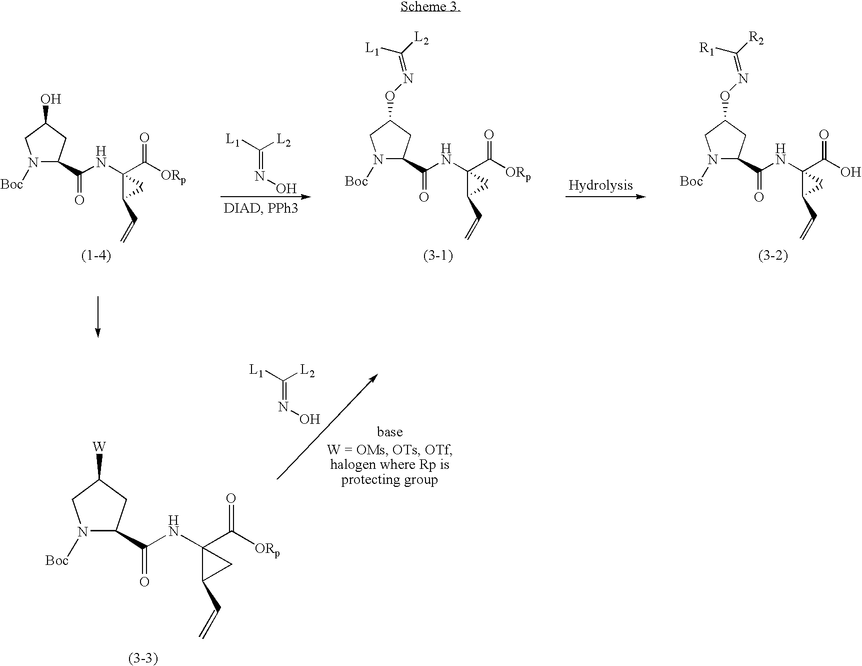

The Scheme 3 describes the alternative methods to synthesize formula (3-2). The intermediates (3-1) can be made directly through (1-4) and oximes using Mitsunobu conditions. Or, intermediate (3-1) can also be made through SN2 replacement of activated hydroxyl group by converting hydroxy intermediate (1-4) to a suitable leaving group such as, but not limited to OMs, OTs, OTf, bromide, or iodide. Subsequent removal of the acid protecting group furnishes compounds of formula (3-2).

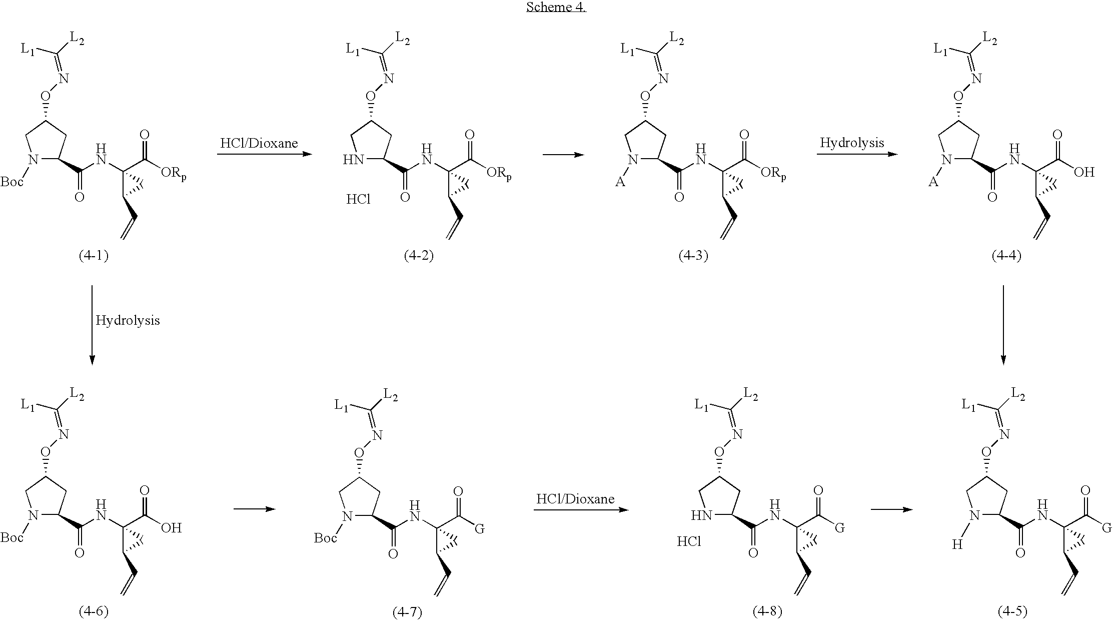

Scheme 4 illustrates the modification of the N-terminal and C-terminal of the acyclic peptide (4-1). Deprotection of the Boc moiety with an acid, such as, but not limited to hydrochloric acid yields compounds of formula (4-2). The amino moiety of formula (4-2) can be alkylated or acylated with appropriate alkyl halide or acyl groups to give compounds of formula (4-3). Compounds of formula (4-3) can be hydrolyzed with base such as lithium hydroxide to free up the acid moiety of formula (4-4). Subsequent activation of the acid moiety followed by treatment with appropriate amino groups, such as, but not limited to amides, or sulfonamides to provide compounds of formula (4-5). Alternatively, the acyclic peptide (4-1) can be hydrolyzed to give the acid (4-6). Subsequently the acid can be converted to compounds of formula (4-7). Deprotection of the Boc group and then alkylation or acylation of amino group yield compounds of formula (4-5).

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

### Example 1

### Compound of Formula XII, wherein A=Boc, L₁ and L₂ Taken Together with the Carbon Atom to Which They are Attached are

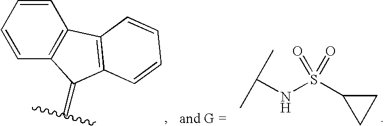

### Step 1a.

To a solution of commercially available Boc-trans-L-hydroxyproline (2.0 g) and D-1-vinyl cyclopropane amino acid ethyl ester (2.0 g) in 15 ml DMF, DIEA (6 ml) and HATU (3.95 g) were added. The coupling was carried out at 0° C. for 1.5 hours. The reaction mixture was diluted with 200 mL EtOAc and subsequently the extract was washed with 5% citric acid (2×20 ml), water (2×20 ml), 1M NaHCO₃ (4×20 ml), and brine (2×10 ml), respectively. The organic phase was dried over anhydrous Na₂SO₄ and evaporated in vacuo, affording dipeptide (3.2 g) which was directly used in the next step.

MS (ESI): m/z=369.23 [M+H].

### Step 1b.

A solution of dipeptide from step 1a (8.65 mmol) in 20 mL DCM was cooled down to −78° C. 2,6-lutidine (2.3 ml) was added and followed by trifluoromethanesulfonyl anhydride (1.6 ml) dropwise. The reaction mixture was kept at −78° C. for 1 hour and then diluted with 300 ml ether. The organic phase was washed with 5% citric acid (3×100 ml) and water. The ether layer was concentrated in vacuo. DMSO/H₂O (20 ml/1 ml) was poured into the residue. The inversion finished in 30 minutes followed by HPLC. The reaction mixture was extracted with 300 mL EtOAc, and washed with brine (3×100 ml), respectively. The organic phase was dried over anhydrous Na₂SO₄ and then concentrated in vacuo. The residue was purified by silica gel flash chromatography using different ratios of hexanes:EtOAc as elution phase (5:1→3:1→1:1→1:2). The desired cis dipeptide was isolated as oil after removal of the elution solvents (2.0 g, 65%).

MS (ESI): m/z=369.23[M+H].

### Step 1c.

To a solution of the dipeptide precursor from step 1b (1.0 g, 2.72 mmol) and DIEA (1.42 ml, 8.16 mmol) in 10.0 ml DCM, mesylate chloride (0.318 ml, 4.08 mmol) was added slowly at 0° C. where the reaction was kept for 3 hours. 100 mL EtOAc was then added and followed by washing with 5% citric acid 2×20 ml, water 1×20 ml, 1M NaHCO₃ 2×20 ml and brine 1×20 ml, respectively. The organic phase was dried over anhydrous Na₂SO₄, filtered and concentrated, yielding the title compound mesylate (1.2 g) that was used for next step synthesis without further purification.

MS (ESI): m/z=447.25 [M+H].

### Step 1d.

To a solution of the mesylate from step 1b (800 mg) in 5 mL DMF, was added 525 mg of 9-Fluorenone oxime and anhydrous cesium carbonate (1.75 g). The resulting reaction mixture was stirred vigorously at 50° C. for 12 hours. The reaction mixture was extracted with EtOAc. The organic layer was washed with 1M NaHCO₃, water, brine, dried over Na₂SO₄, filtered and concentrated. The residue was purified by silica gel chromatography to give 760 mg of desired product.

MS (ESI): m/z=546.32 [M+H].

### Step 1e.

The compound from step 1d was hydrolyzed with LiOH in THF/MeOH/H₂O (2:1:1) overnight. The reaction mixture was acidified with 1N HCl, extracted with 3 mL EtOAc, and washed with brine 2×1 ml. The organic phase was dried over anhydrous Na₂SO₄ and then evaporated to give desired acid (660 mg) without further purification.

MS (ESI): m/z=518.34 [M+H].

### Step 1f.

To a solution of the compound (460 mg) from step 1e in DCM was added CDI (202 mg). The reaction mixture was stirred at 40° C. for 1 h and then added cyclopropylsulfonamide (269 mg) and DBU (2671). The reaction mixture was stirred overnight at 40° C. The reaction mixture was extracted with EtOAc. The organic extracts were washed with 1M NaHCO₃, brine, dried over Na₂SO₄, filtered and concentrated. The residue was purified by silica gel chromatograph to give desired product (570 mg).

MS (ESI): m/z=621.41 [M+H].

### Example 2

### Compound of Formula XII, Wherein A=H, L₁ and L₂ Taken Together with the Carbon Atom to Which They are Attached are

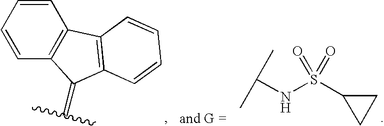

To a flask containing the compound from step 1f (200 mg) of Example 1 was added 4N HCl/dioxane (25 ml). The resulting mixture was stirred for 1 hr at room temperature. The mixture was then concentrated to give desired product without further purification.

MS (ESI): m/z=521.26 [M+H].

### Example 3

### Compound of Formula XII, Wherein

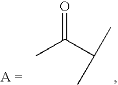

### L₁ and L₂ Taken Together with the Carbon Atom to Which They are Attached are

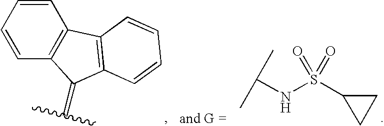

To a solution of the compound from Example 2 (0.032 mmol) in DCM was added DIEA (28 μl) and acetyl anhydride (6 μl) at 0° C. The mixture was stirred overnight at room temperature. The reaction mixture was extracted with EtOAc. The organic extracts were washed with NaHCO₃, brine, dried over Na₂SO₄, filtered and concentrated. The crude product was purified by HPLC to give the desired product.

MS (ESI): m/z=563.19 [M+H].

### Example 4

### Compound of Formula XII, Wherein

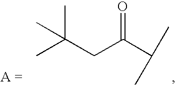

### L₁ and L₂ Taken Together with the Carbon Atom to Which They are Attached are

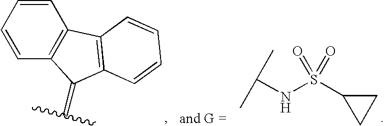

To a solution of the compound from Example 2 (0.032 mmol) in acetonitrile (2 ml) was added tert-Butyl acetic acid (7.5 mg), HATU (27 mg) and DIEA (28 μl) at 0° C. The mixture was stirred overnight at room temperature. The reaction mixture was extracted with EtOAc. The organic extracts were washed with NaHCO₃, brine, dried over Na₂SO₄, filtered and concentrated. The crude product was purified by HPLC to give the desired product.

MS (ESI): m/z=619.24 [M+H].

Example 5 to Example 20 (Formula XII) was made following the procedures described in Examples 4.

### Example 5

### Compound of Formula XII, Wherein

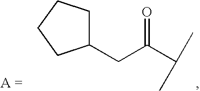

### L₁ and L₂ Taken Together with the carbon atom to which they are attached are

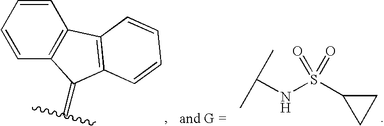

MS (ESI): m/z=631.27 [M+H].

### Example 6

### Compound of Formula XII, Wherein

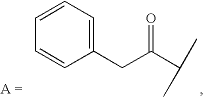

### L₁ and L₂ Taken Together with the Carbon Atom to Which They are Attached are

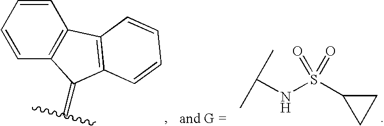

MS (ESI): m/z=639.22 [M+H].

### Example 7

### Compound of Formula XII, Wherein

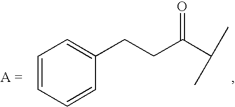

### L₁ and L₂ Taken Together with the Carbon Atom to Which They are Attached are

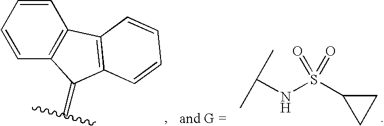

MS (ESI): m/z=653.23 [M+H].

### Example 8

### Compound of Formula XII, Wherein

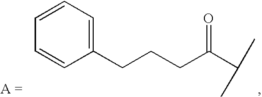

### L₁ and L₂ Taken Together with the Carbon Atom to Which They are Attached are

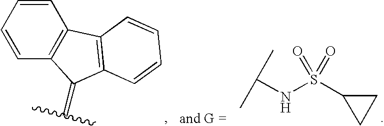

MS (ESI): m/z=667.25 [M+H].

### Example 9

### Compound of Formula XII, Wherein

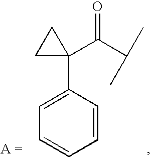

### L₁ and L₂ Taken Together with the Carbon Atom to Which They are Attached are

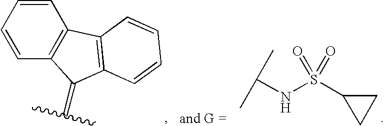

MS (ESI): m/z=665.26 [M+H].

### Example 10

### Compound of Formula XII, Wherein

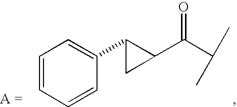

### L₁ and L₂ Taken Together with the Carbon Atom to Which They are Attached are

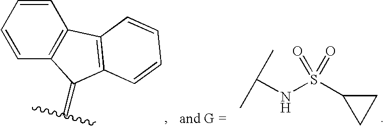

MS (ESI): m/z=665.29 [M+H].

### Example 11

### Compound of Formula XII, Wherein

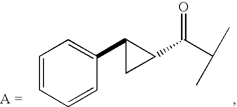

### L₁ and L₂ Taken Together with the Carbon Atom to Which They are Attached are

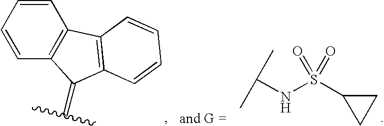

MS (ESI): m/z=665.29 [M+H].

### Example 12

### Compound of Formula XII, Wherein

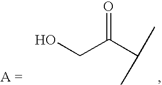

### L₁ and L₂ Taken Together with the Carbon Atom to Which They are Attached are

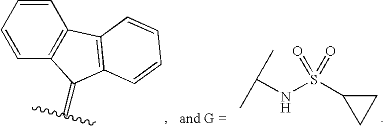

MS (ESI): m/z=579.23 [M+H].

### Example 13

### Compound of Formula XII, Wherein

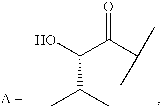

### L₁ and L₂ Taken Together with the Carbon Atom to Which They are Attached are

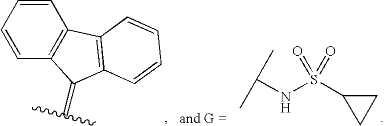

MS (ESI): m/z=621.27 [M+H].

### Example 14

### Compound of Formula XII, Wherein

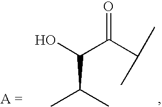

### L₁ and L₂ taken together with the Carbon Atom to Which They are Attached are

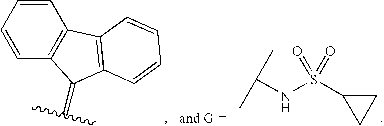

MS (ESI): m/z=621.27 [M+H].

### Example 15

### Compound of Formula XII, Wherein

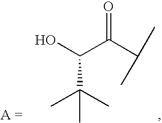

### L₁ and L₂ taken together with the Carbon Atom to Which They are Attached are

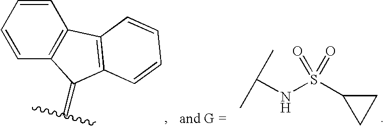

MS (ESI): m/z=635.26 [M+H].

### Example 16

### Compound of Formula XII, Wherein

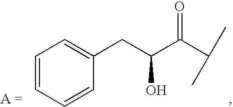

### L₁ and L₂ Taken Together with the Carbon Atom to Which They are Attached are

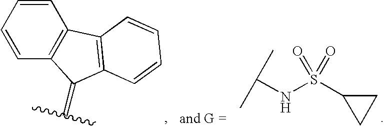

MS (ESI): m/z=669.25 [M+H].

### Example 17

### Compound of Formula XII, Wherein

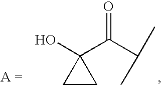

### L₁ and L₂ Taken Together with the Carbon Atom to Which They are Attached are

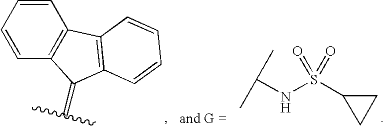

MS (ESI): m/z=605.19 [M+H].

### Example 18

### Compound of Formula XII, Wherein

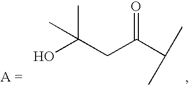

### L₁ and L₂ Taken Together with the Carbon Atom to Which They are Attached are

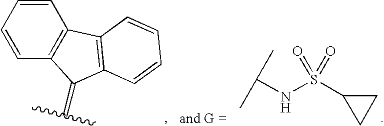

MS (ESI): m/z=621.24 [M+H].

### Example 19

### Compound of Formula XII, Wherein

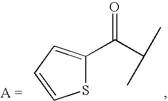

### L₁ and L₂ Taken Together with the Carbon Atom to Which They are Attached are

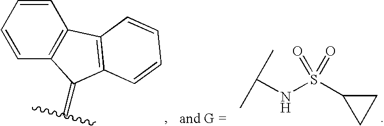

MS (ESI): m/z=631.16 [M+H].

### Example 20

### Compound of Formula XII, Wherein

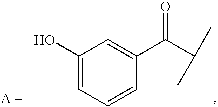

### L₁ and L₂ Taken Together with the Carbon Atom to Which They are Attached are

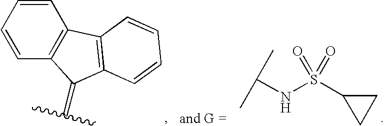

MS (ESI): m/z=641.22 [M+H].

### Example 21

### Compound of Formula XII, Wherein

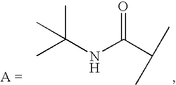

### L₁ and L₂ Taken Together with the Carbon Atom to Which They are Attached are

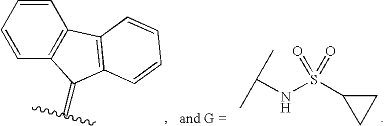

To a solution of the compound from Example 2 (0.032 mmol) in DCM was added DIEA (28 μl) and tert-butylisocyanate (7.5 μl) at 0° C. The mixture was stirred for 1.5 h at room temperature. The reaction mixture was extracted with EtOAc. The organic extracts were washed with NaHCO₃, brine, dried over Na₂SO₄, filtered and concentrated. The crude product was purified by HPLC to give desired product.

MS (ESI): m/z=620.34 [M+H].

### Example 22

### Compound of Formula XII, Wherein

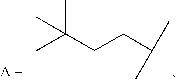

### L₁ and L₂ Taken Together with the Carbon Atom to Which They are Attached are

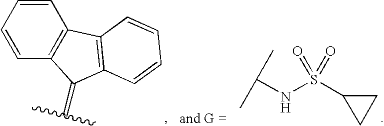

To a solution of the compound from Example 2 (0.032 mmol) in acetonitrile was added acetic acid (5 μl), 3,3-Dimethyl-butyraldehyde (201) and sodium cyanoborohydride (4 mg) at RT. The mixture was stirred for 1.5 h at room temperature. The reaction mixture was extracted with EtOAc. The organic extracts were washed with NaHCO₃, brine, dried over Na₂SO₄, filtered and concentrated. The crude product was purified by HPLC to give desired product.

MS (ESI): m/z=605.29 [M+H].

### Example 23

### Compound of Formula XII, Wherein

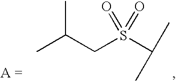

### L₁ and L₂ Taken Together with the Carbon Atom to Which They are Attached are

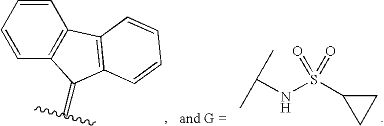

To a solution of the compound from Example 2 (0.032 mmol) in acetonitrile was added DIEA (28 μl), isobutanesulfonylchloride (6.5 μl) and DMAP (1 mg) at RT. The mixture was stirred for 3 h at room temperature. The reaction mixture was extracted with EtOAc. The organic extracts were washed with NaHCO₃, brine, dried over Na₂SO₄, filtered and concentrated. The crude product was purified by HPLC to give desired product.

MS (ESI): m/z=641.22 [M+H].

### Example 24

### Compound of Formula XII, Wherein

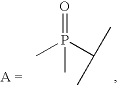

### L₁ and L₂ Taken Together with the Carbon Atom to Which They are Attached are

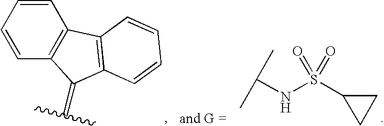

To a solution of the compound from Example 2 (0.032 mmol) in DCM was added DIEA (28 μl) and Dimethylphosphinic chloride (5.4 mg) RT. The mixture was stirred for 3 h at room temperature. The reaction mixture was extracted with EtOAc. The organic extracts were washed with NaHCO₃, brine, dried over Na₂SO₄, filtered and concentrated. The crude product was purified by HPLC to give desired product.

MS (ESI): m/z=597.21 [M+H].

Example 25 to Example 160 (Formula XII) are made following the procedures described in Examples 1-24.

TABLE 1

| Example | A | L₁L₂ | G |
|---|---|---|---|
| 25 | 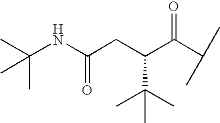 | 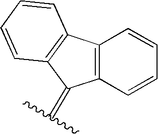 | 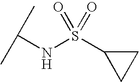 |
| 26 | 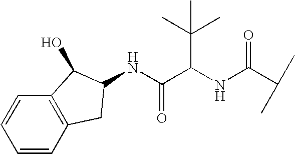 | 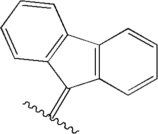 | 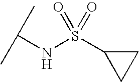 |
| 27 | 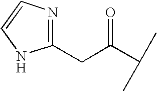 | 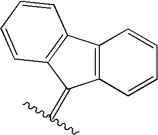 | 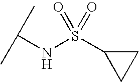 |
| 28 | 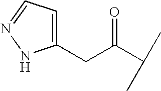 | 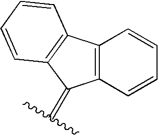 | 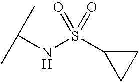 |
| 29 | 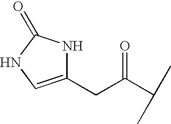 | 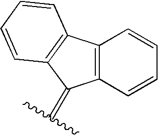 | 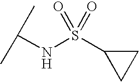 |
| 30 | 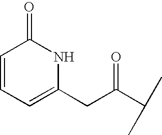 | 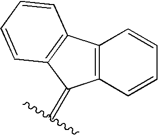 | 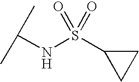 |
| 31 | 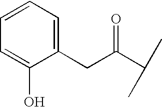 | 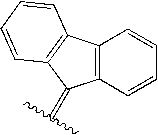 | 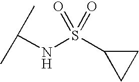 |
| 32 | 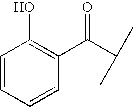 | 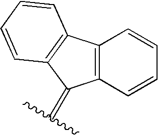 | 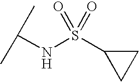 |
| 33 | 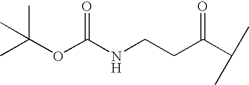 | 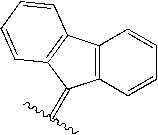 | 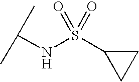 |
| 34 | 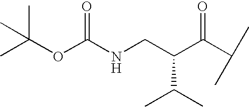 | 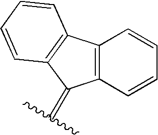 | 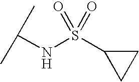 |
| 35 | 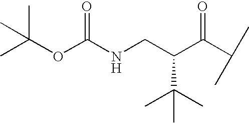 | 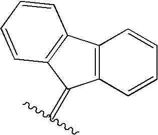 | 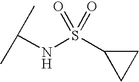 |
| 36 | 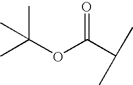 | 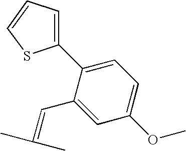 | 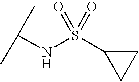 |
| 37 | 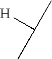 | 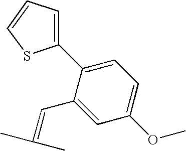 | 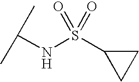 |
| 38 | 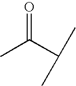 | 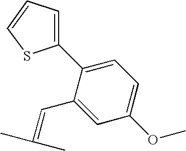 | 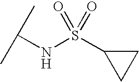 |
| 39 | 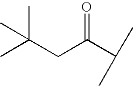 | 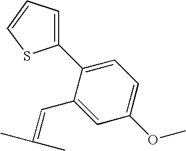 | 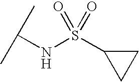 |
| 40 | 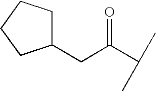 | 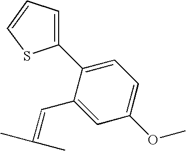 | 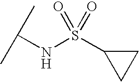 |
| 41 | 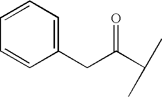 | 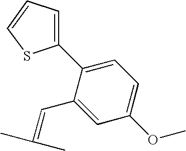 | 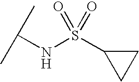 |
| 42 | 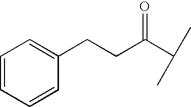 | 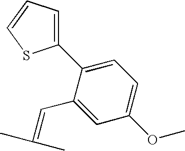 | 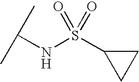 |
| 43 | 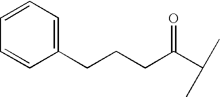 | 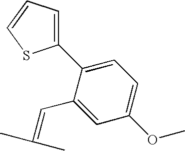 | 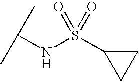 |
| 44 | 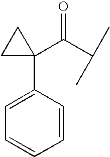 | 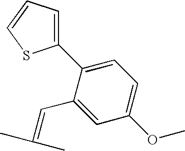 | 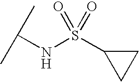 |
| 45 | 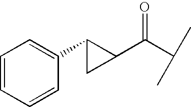 | 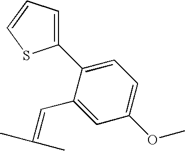 | 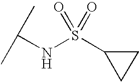 |
| 46 | 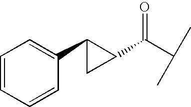 | 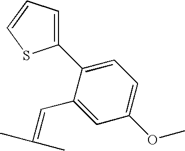 | 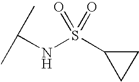 |
| 47 | 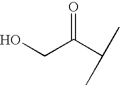 | 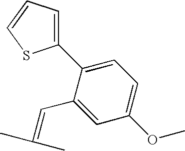 | 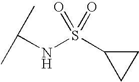 |
| 48 | 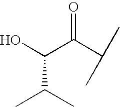 | 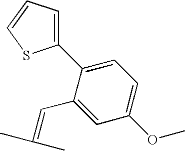 | 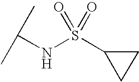 |
| 49 | 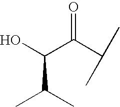 | 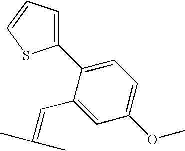 | 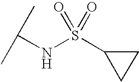 |
| 50 | 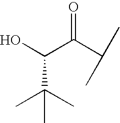 | 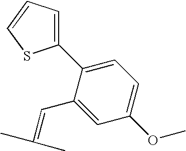 | 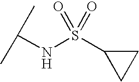 |
| 51 | 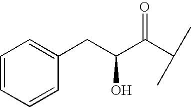 | 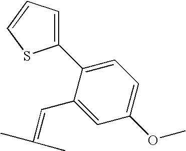 | 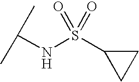 |
| 52 | 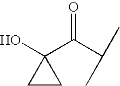 | 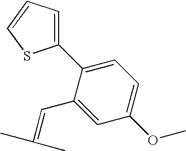 | 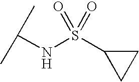 |
| 53 | 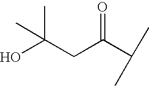 | 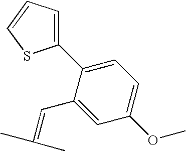 | 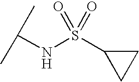 |
| 54 | 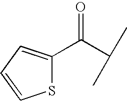 | 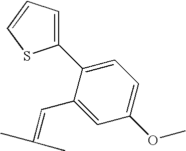 | 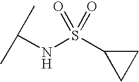 |
| 55 | 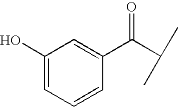 | 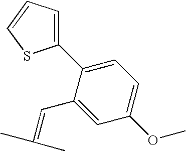 | 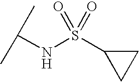 |
| 56 | 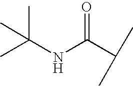 | 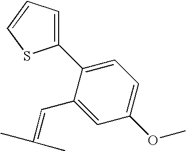 | 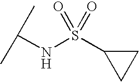 |
| 57 | 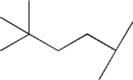 | 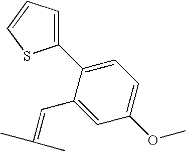 | 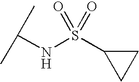 |
| 58 | 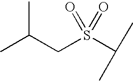 | 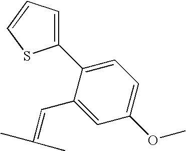 | 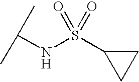 |
| 59 | 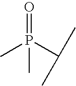 | 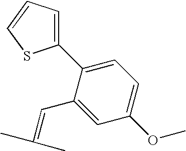 | 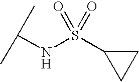 |
| 60 | 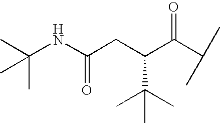 | 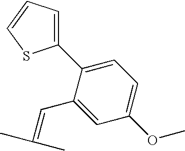 | 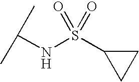 |
| 61 | 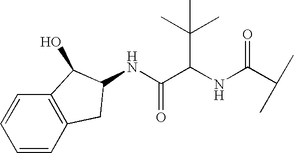 | 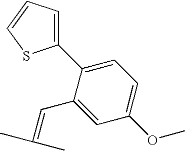 | 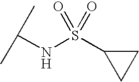 |
| 62 | 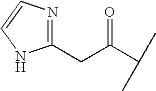 | 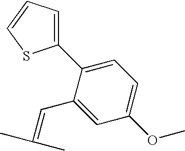 | 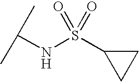 |
| 63 | 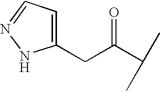 | 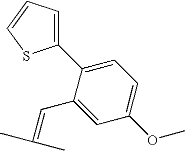 | 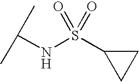 |
| 64 | 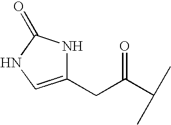 | 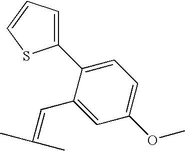 | 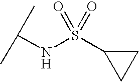 |
| 65 | 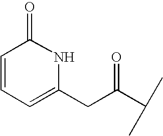 | 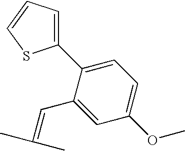 | 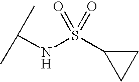 |
| 66 | 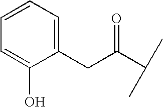 | 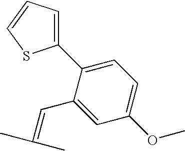 | 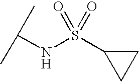 |
| 67 | 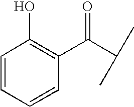 | 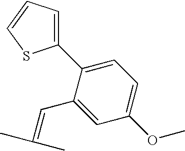 | 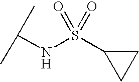 |
| 68 | 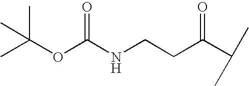 | 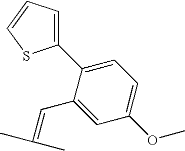 | 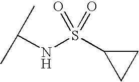 |
| 69 | 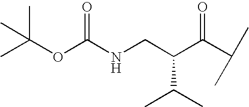 | 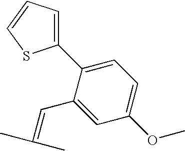 | 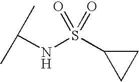 |
| 70 | 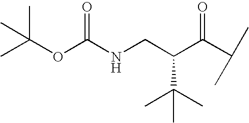 | 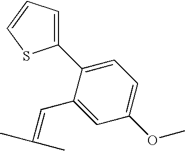 | 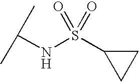 |
| 71 | 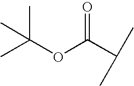 | 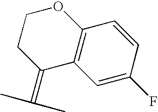 | 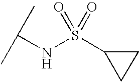 |
| 72 | 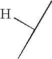 | 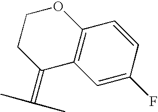 | 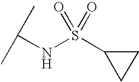 |
| 73 | 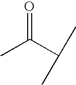 | 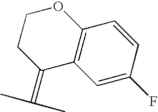 | 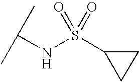 |
| 74 | 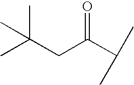 | 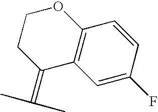 | 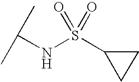 |
| 75 | 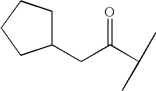 | 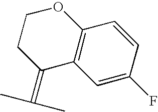 | 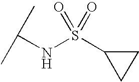 |
| 76 | 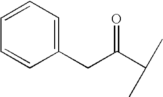 | 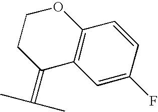 | 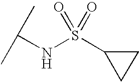 |
| 77 | 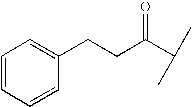 | 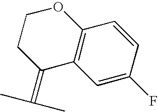 | 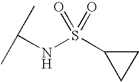 |
| 78 | 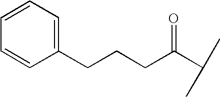 | 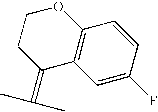 | 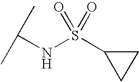 |
| 79 | 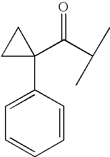 | 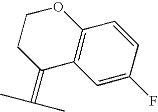 | 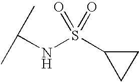 |
| 80 | 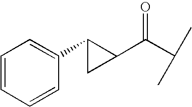 | 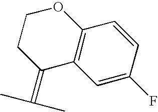 | 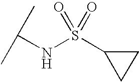 |
| 81 | 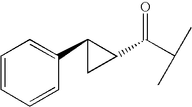 | 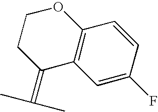 | 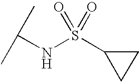 |
| 82 | 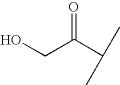 | 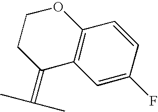 | 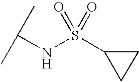 |
| 83 | 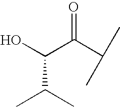 | 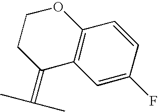 | 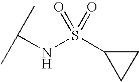 |
| 84 | 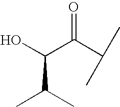 | 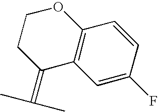 | 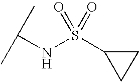 |
| 85 | 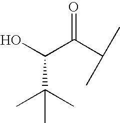 | 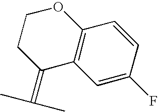 | 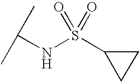 |
| 86 | 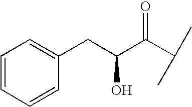 | 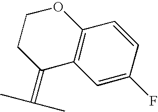 | 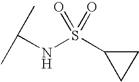 |
| 87 | 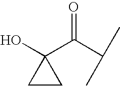 | 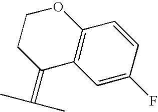 | 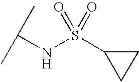 |
| 88 | 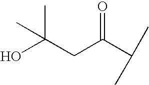 | 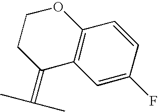 | 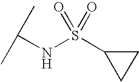 |
| 89 | 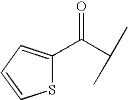 | 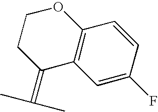 | 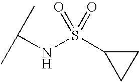 |
| 90 | 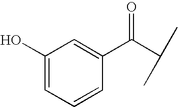 | 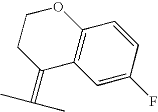 | 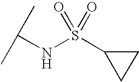 |
| 91 | 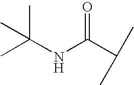 | 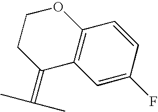 | 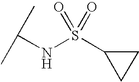 |
| 92 | 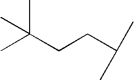 | 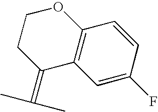 | 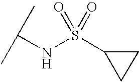 |
| 93 | 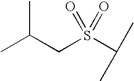 | 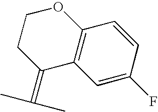 | 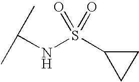 |
| 94 | 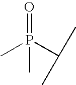 | 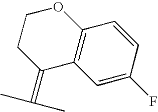 | 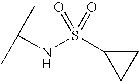 |
| 95 | 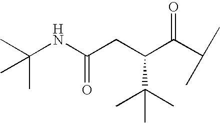 | 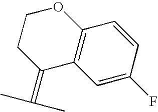 | 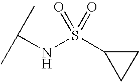 |
| 96 | 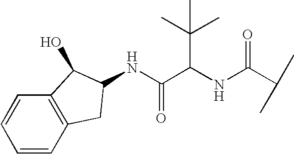 | 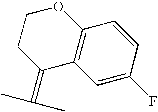 | 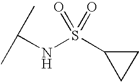 |
| 97 | 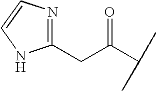 | 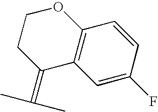 | 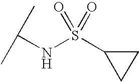 |
| 98 | 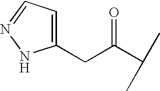 | 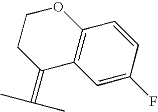 | 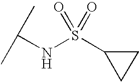 |
| 99 | 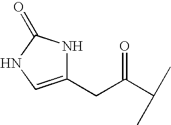 | 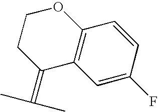 | 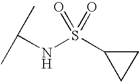 |
| 100 | 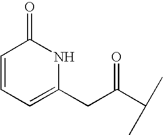 | 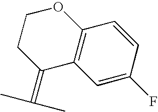 | 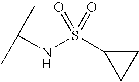 |
| 101 | 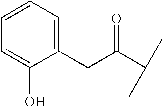 | 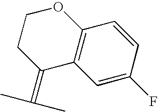 | 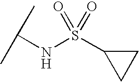 |
| 102 | 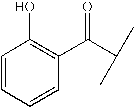 | 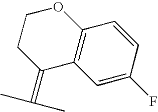 | 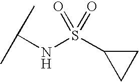 |
| 103 | 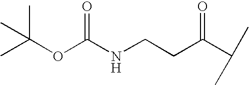 | 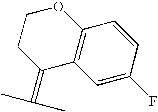 | 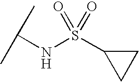 |
| 104 | 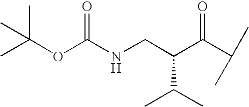 | 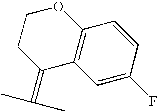 | 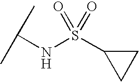 |
| 105 | 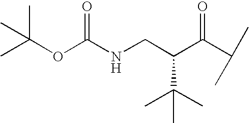 | 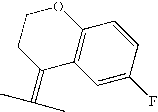 | 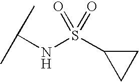 |
| 106 | 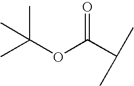 | 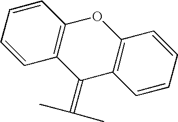 | 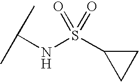 |
| 107 | 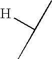 | 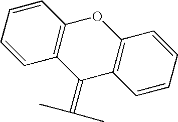 | 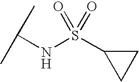 |
| 108 | 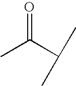 | 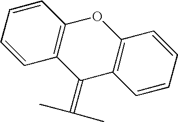 | 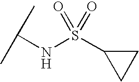 |
| 109 | 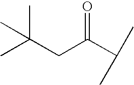 | 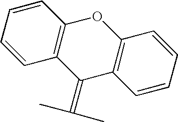 | 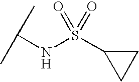 |
| 110 | 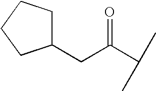 | 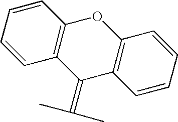 | 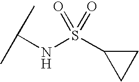 |
| 111 | 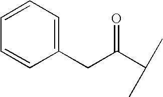 | 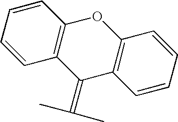 | 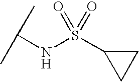 |
| 112 | 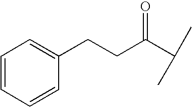 | 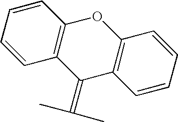 | 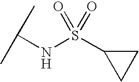 |
| 113 | 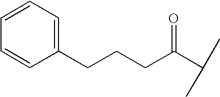 | 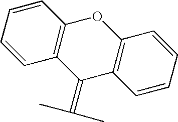 | 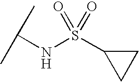 |
| 114 | 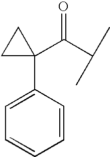 | 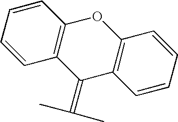 | 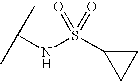 |
| 115 | 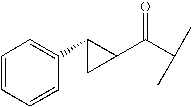 | 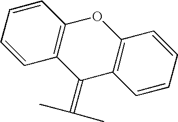 | 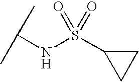 |
| 116 | 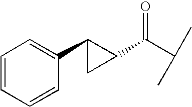 | 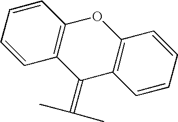 | 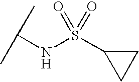 |
| 117 | 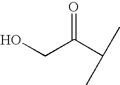 | 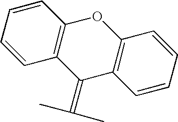 | 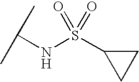 |
| 118 | 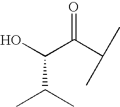 | 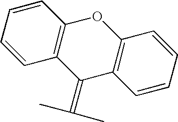 | 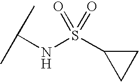 |
| 119 | 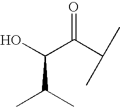 | 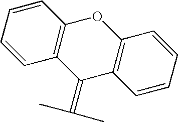 | 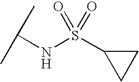 |
| 120 | 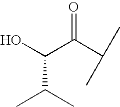 | 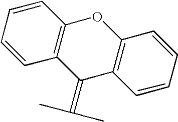 | 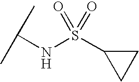 |
| 121 | 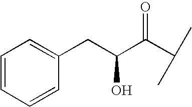 | 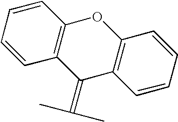 | 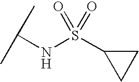 |
| 122 | 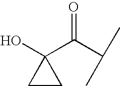 | 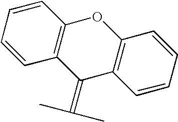 | 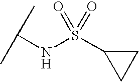 |
| 123 | 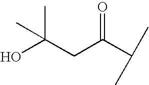 | 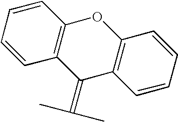 | 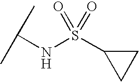 |
| 124 | 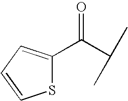 | 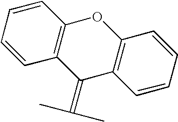 | 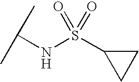 |
| 125 | 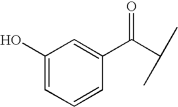 | 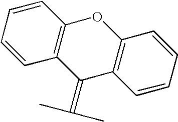 | 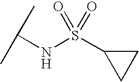 |
| 126 | 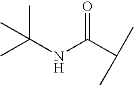 | 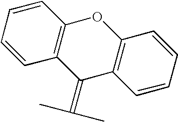 | 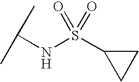 |
| 127 | 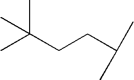 | 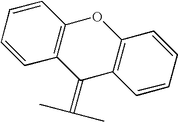 | 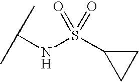 |
| 128 | 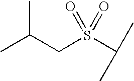 | 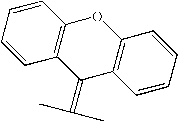 | 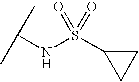 |
| 129 | 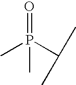 | 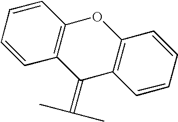 | 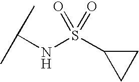 |
| 130 | 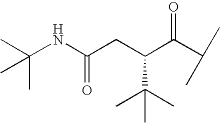 | 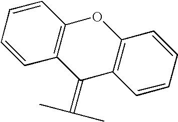 | 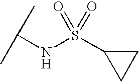 |
| 131 | 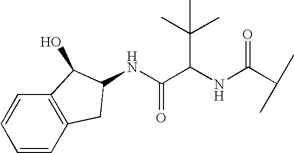 | 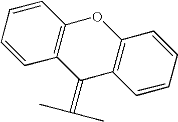 | 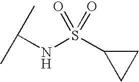 |
| 132 | 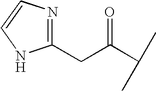 | 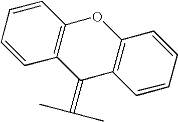 | 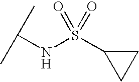 |
| 133 | 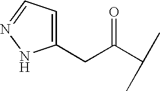 | 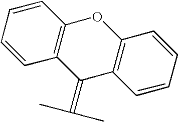 | 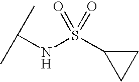 |
| 134 | 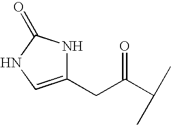 | 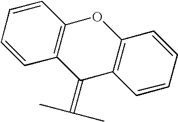 | 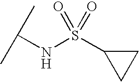 |
| 135 | 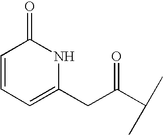 | 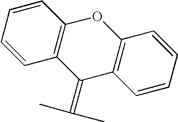 | 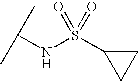 |
| 136 | 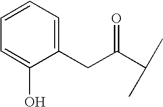 | 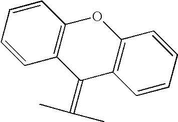 | 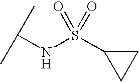 |
| 137 | 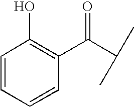 | 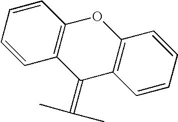 | 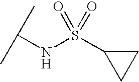 |
| 138 | 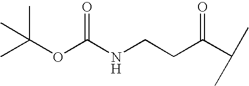 | 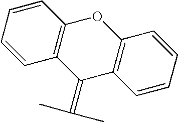 | 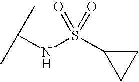 |
| 139 | 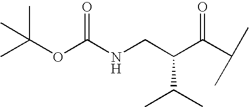 | 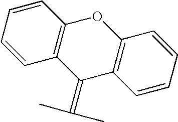 | 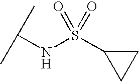 |
| 140 | 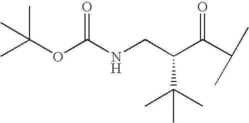 | 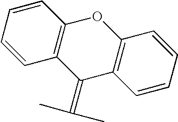 | 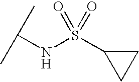 |
| 141 | 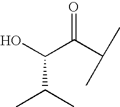 | 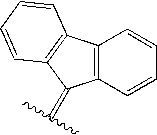 | 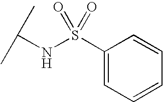 |
| 142 | 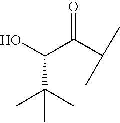 | 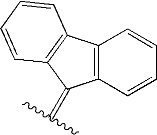 | 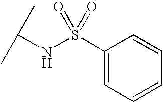 |
| 143 | 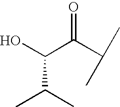 | 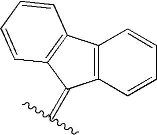 | 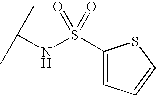 |
| 144 | 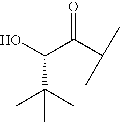 | 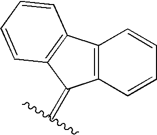 | 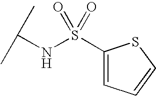 |
| 145 | 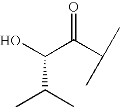 | 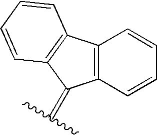 | 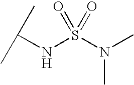 |
| 146 | 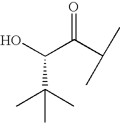 | 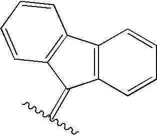 | 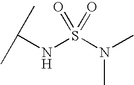 |
| 147 | 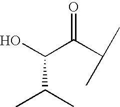 | 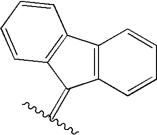 | 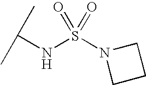 |
| 148 | 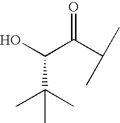 | 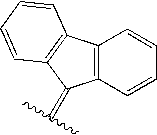 | 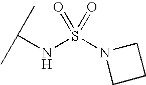 |
| 149 | 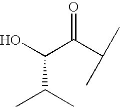 | 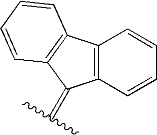 | 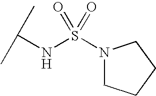 |
| 150 | 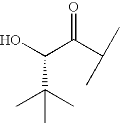 | 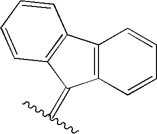 | 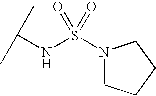 |
| 151 | 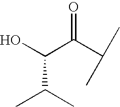 | 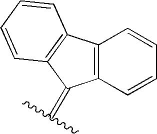 | 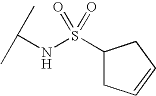 |
| 152 | 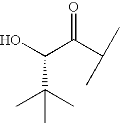 | 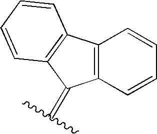 | 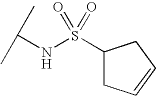 |
| 153 | 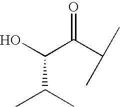 | 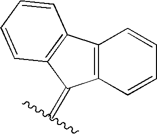 | 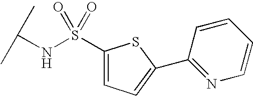 |
| 154 | 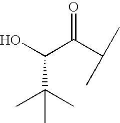 | 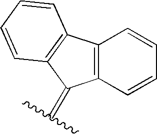 | 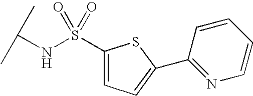 |
| 155 | 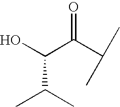 | 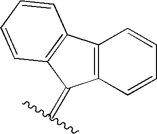 | 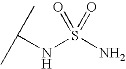 |
| 156 | 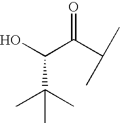 | 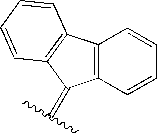 | 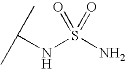 |
| 157 | 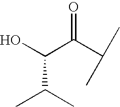 | 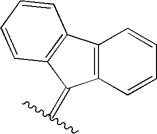 | 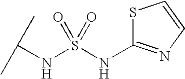 |
| 158 | 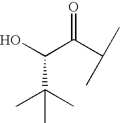 | 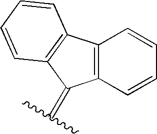 | 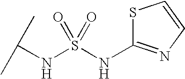 |
| 159 | 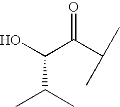 | 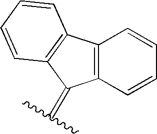 | 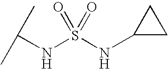 |
| 160 | 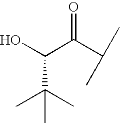 | 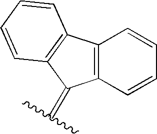 | 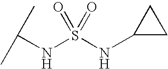 |

The compounds of the present invention exhibit potent inhibitory properties against the HCV NS3 protease. The following examples describe assays in which the compounds of the present invention can be tested for anti-HCV effects.

### Example 161

### NS3/NS4a Protease Enzyme Assay

HCV protease activity and inhibition is assayed using an internally quenched fluorogenic substrate. A DABCYL and an EDANS group are attached to opposite ends of a short peptide. Quenching of the EDANS fluorescence by the DABCYL group is relieved upon proteolytic cleavage. Fluorescence is measured with a Molecular Devices Fluoromax (or equivalent) using an excitation wavelength of 355 nm and an emission wavelength of 485 nm.

The assay is run in Corning white half-area 96-well plates (VWR 29444-312 [Corning 3693]) with full-length NS3 HCV protease 1b tethered with NS4A cofactor (final enzyme concentration 1 to 15 nM). The assay buffer is complemented with 10 μM NS4A cofactor Pep 4A (Anaspec 25336 or in-house, MW 1424.8). RET S1 (Ac-Asp-Glu-Asp(EDANS)-Glu-Glu-Abu-[COO]Ala-Ser-Lys-(DABCYL)-NH₂, AnaSpec 22991, MW 1548.6) is used as the fluorogenic peptide substrate. The assay buffer contains 50 mM Hepes at pH 7.5, 30 mM NaCl and 10 mM BME. The enzyme reaction is followed over a 30 minutes time course at room temperature in the absence and presence of inhibitors.

The peptide inhibitors HCV Inh 1 (Anaspec 25345, MW 796.8) Ac-Asp-Glu-Met-Glu-Glu-Cys-OH, [−20° C.] and HCV Inh 2 (Anaspec 25346, MW 913.1) Ac-Asp-Glu-Dif-Cha-Cys-OH, are used as reference compounds.

IC₅₀ values are calculated using XLFit in ActivityBase (IDBS) using equation 205:

y=A+((B−A)/(1+((C/x)̂D))).

### Example 162

### Cell-Based Replicon Assay

Quantification of HCV replicon RNA in cell lines (HCV Cell Based Assay) Cell lines, including Huh-11-7 or Huh 9-13, harboring HCV replicons (Lohmann, et al Science 285:110-113, 1999) are seeded at 5×10³ cells/well in 96 well plates and fed media containing DMEM (high glucose), 10% fetal calf serum, penicillin-streptomycin and non-essential amino acids. Cells are incubated in a 7.5% CO₂ incubator at 37° C. At the end of the incubation period, total RNA is extracted and purified from cells using Qiagen Rneasy 96 Kit (Catalog No. 74182). To amplify the HCV RNA so that sufficient material can be detected by an HCV specific probe (below), primers specific for HCV (below) mediate both the reverse transcription of the HCV RNA and the amplification of the cDNA by polymerase chain reaction (PCR) using the TaqMan One-Step RT-PCR Master Mix Kit (Applied Biosystems catalog no. 4309169). The nucleotide sequences of the RT-PCR primers, which are located in the NS5B region of the HCV genome, are the following:

HCV Forward primer “RBNS5bfor”

| | |
|---|---|
| 5′GCTGCGGCCTGTCGAGCT: | (SEQ ID NO: 1) |

HCV Reverse primer “RBNS5Brev”

| | |
|---|---|
| 5′CAAGGTCGTCTCCGCATAC. | (SEQ ID NO 2) |

Detection of the RT-PCR product is accomplished using the Applied Biosystems (ABI) Prism 7500 Sequence Detection System (SDS) that detects the fluorescence that is emitted when the probe, which is labeled with a fluorescence reporter dye and a quencher dye, is processed during the PCR reaction. The increase in the amount of fluorescence is measured during each cycle of PCR and reflects the increasing amount of RT-PCR product. Specifically, quantification is based on the threshold cycle, where the amplification plot crosses a defined fluorescence threshold. Comparison of the threshold cycles of the sample with a known standard provides a highly sensitive measure of relative template concentration in different samples (ABI User Bulletin #2 Dec. 11, 1997). The data is analyzed using the ABI SDS program version 1.7. The relative template concentration can be converted to RNA copy numbers by employing a standard curve of HCV RNA standards with known copy number (ABI User Bulletin #2 Dec. 11, 1997).

The RT-PCR product was detected using the following labeled probe:

(SEQ ID NO: 3)

5′ FAM-CGAAGCTCCAGGACTGCACGATGCT-TAMRA

FAM=Fluorescence reporter dye.
TAMRA:=Quencher dye.

The RT reaction is performed at 48° C. for 30 minutes followed by PCR. Thermal cycler parameters used for the PCR reaction on the ABI Prism 7500 Sequence Detection System are: one cycle at 95° C., 10 minutes followed by 40 cycles each of which include one incubation at 95° C. for 15 seconds and a second incubation for 60° C. for 1 minute.

To normalize the data to an internal control molecule within the cellular RNA, RT-PCR is performed on the cellular messenger RNA glyceraldehydes-3-phosphate dehydrogenase (GAPDH). The GAPDH copy number is very stable in the cell lines used. GAPDH RT-PCR is performed on the same exact RNA sample from which the HCV copy number is determined. The GAPDH primers and probes, as well as the standards with which to determine copy number, are contained in the ABI Pre-Developed TaqMan Assay Kit (catalog no. 4310884E). The ratio of HCV/GAPDH RNA is used to calculate the activity of compounds evaluated for inhibition of HCV RNA replication.

### Activity of Compounds as Inhibitors of HCV Replication (Cell Based Assay) In Replicon Containing Huh-7 Cell Lines

The effect of a specific anti-viral compound on HCV replicon RNA levels in Huh-11-7 or 9-13 cells is determined by comparing the amount of HCV RNA normalized to GAPDH (e.g. the ratio of HCV/GAPDH) in the cells exposed to compound versus cells exposed to the 0% inhibition and the 100% inhibition controls. Specifically, cells are seeded at 5×10³ cells/well in a 96 well plate and are incubated either with: 1) media containing 1% DMSO (0% inhibition control), 2) 100 international units, IU/ml Interferon-alpha 2b in media/1% DMSO or 3) media/1% DMSO containing a fixed concentration of compound. 96 well plates as described above are then incubated at 37° C. for 3 days (primary screening assay) or 4 days (IC50 determination). Percent inhibition is defined as:

% Inhibition=[100−((S-C2)/C₁-C₂))]×100

where
S=the ratio of HCV RNA copy number/GAPDH RNA copy number in the sample;
C₁=the ratio of HCV RNA copy number/GAPDH RNA copy number in the 0% inhibition control (media/1% DMSO); and
C2=the ratio of HCV RNA copy number/GAPDH RNA copy number in the 100% inhibition control (100 IU/ml Interferon-alpha 2b).

The dose-response curve of the inhibitor is generated by adding compound in serial, three-fold dilutions over three logs to wells starting with the highest concentration of a specific compound at 10 uM and ending with the lowest concentration of 0.01 uM. Further dilution series (1 uM to 0.001 uM for example) is performed if the IC50 value is not in the linear range of the curve. IC50 is determined based on the IDBS Activity Base program using Microsoft Excel “XL Fit” in which A=100% inhibition value (100IU/ml Interferon-alpha 2b), B=0% inhibition control value (media/1% DMSO) and C=midpoint of the curve as defined as C=(B−A/2)+A. A, B and C values are expressed as the ratio of HCV RNA/GAPDH RNA as determined for each sample in each well of a 96 well plate as described above. For each plate the average of 4-6 wells are used to define the 100% and 0% inhibition values.

In the above assays, representative compounds are found to have activity.

Although the invention has been described with respect to various preferred embodiments, it is not intended to be limited thereto, but rather those skilled in the art will recognize that variations and modifications may be made therein which are within the spirit of the invention and the scope of the appended claims.

What is claimed is:

1. A compound represented by the formula I: 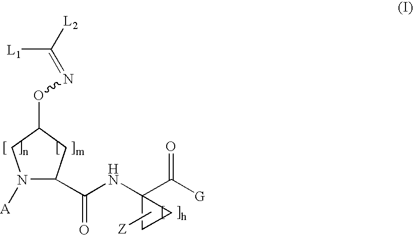
and pharmaceutically acceptable salts, esters and prodrugs thereof, wherein:
A is selected from the group consisting of:
(1) R1;
(2) (CO)R₁;
(3) (CO)OR₁;
(4) (CO)NR₁R₂;
(5) SO₂R₁;
(6) (SO₂)OR₁;
(7) SO₂NR₁R₂;
(8) (C═NR₁)NR₂R₃;
(9) (PO)R₁R₂;
(10) (PO)OR10R₂;
(11) (PO)NRINR₂;
(12) (PO)NR₁OR₂
R₁ and R₂ are independently selected from the group consisting of:
a) hydrogen;
b) aryl;
c) substituted aryl;
d) heteroaryl;
e) substituted heteroaryl;
f) heterocyclic or substituted heterocyclic;
g) —C₁-C₈ alkyl, —C₂-C₈ alkenyl, or —C₂-C₈ alkynyl containing 0, 1, 2, or 3 heteroatoms selected from O, S or N;
h) substituted —C₁-C₈ alkyl, substituted —C₂-C₈ alkenyl, or substituted —C₂-C₈ alkynyl containing 0, 1, 2, or 3 heteroatoms selected from O, S or N;
i) —C₃-C₁₂ cycloalkyl;
j) substituted —C₃-C₁₂ cycloalkyl;
k) —C₃-C₁₂ cycloalkenyl;
l) substituted —C₃-C₁₂ cycloalkenyl;
or R₁ and R₂ taken together with the nitrogen atom to which they are attached form a substituted or unsubstituted heterocyclic;
L₁ and L₂ are independently selected from the group consisting of:
(1) hydrogen;
(2) aryl;
(3) substituted aryl;
(4) heteroaryl;
(5) substituted heteroaryl;
(6) heterocyclic or substituted heterocyclic;
(7) —C₁-C₈ alkyl, —C₂-C₈ alkenyl, or —C₂-C₈ alkynyl containing 0, 1, 2, or 3 heteroatoms selected from O, S or N;
(8) substituted —C₁-C₈ alkyl, substituted —C₂-C₈ alkenyl, or substituted —C₂-C₈ alkynyl containing 0, 1, 2, or 3 heteroatoms selected from O, S or N;
(9) —C₃-C₁₂ cycloalkyl;
(10) substituted —C₃-C₁₂ cycloalkyl;
(11) —C₃-C₁₂ cycloalkenyl;
(12) substituted —C₃-C₁₂ cycloalkenyl;
(13)-Q-R₄, where Q is (CO), (CO)O, (CO)NR₅, (SO), (SO₂), (SO₂)NR₅; and R₄ and R₅ are independently selected from the group consisting of:
a) hydrogen;
b) aryl;
c) substituted aryl;
d) heteroaryl;
e) substituted heteroaryl;
f) heterocyclic or substituted heterocyclic;
g) —C₁-C₈ alkyl, —C₂-C₈ alkenyl, or —C₂-C₈ alkynyl containing 0, 1, 2, or 3 heteroatoms selected from O, S or N;
h) substituted —C₁-C₈ alkyl, substituted —C₂-C₈ alkenyl, or substituted —C₂-C₈ alkynyl containing 0, 1, 2, or 3 heteroatoms selected from O, S or N;
i) —C₃-C₁₂ cycloalkyl;
j) substituted —C₃-C₁₂ cycloalkyl;
k) —C₃-C₁₂ cycloalkenyl;
l) substituted —C₃-C₁₂ cycloalkenyl;
or L₁ and L₂ taken together with the carbon atom to which they are attached form a cyclic moiety selected from: substituted or unsubstituted cycloalkyl, cycloalkenyl, or heterocylic; substituted or unsubstituted cycloalkyl, cycloalkenyl, and heterocyclic fused with one or more R₄; where R₄ is as previously defined;
G is -E-R₄; and where E is absent, or E is O, CO, (CO)O, (CO)NR₅, NH, NH(CO), NH(CO)NR₅, NH(SO₂)NR₅ or NHSO₂; where R₄ and R₅ are as previously defined; or R₄ and R₅ taken together with the nitrogen atom to which they are attached form a substituted or unsubstituted heterocylic;
Z is independently selected from the group consisting of:
(1) hydrogen;
(2) aryl;
(3) substituted aryl;
(4) heteroaryl;
(5) substituted heteroaryl;
(6) heterocyclic or substituted heterocyclic;
(7) —C₁-C₈ alkyl, —C₂-C₈ alkenyl, or —C₂-C₈ alkynyl containing 0, 1, 2, or 3 heteroatoms selected from O, S or N;
(8) substituted —C₁-C₈ alkyl, substituted —C₂-C₈ alkenyl, or substituted —C₂-C₈ alkynyl containing 0, 1, 2, or 3 heteroatoms selected from O, S or N;
(9) —C₃-C₁₂ cycloalkyl;
(10) substituted —C₃-C₁₂ cycloalkyl;
(11) —C₃-C₁₂ cycloalkenyl;
(12) substituted —C₃-C₁₂ cycloalkenyl;
h=0, 1, 2 or 3;
m=0, 1, 2 or 3;
n=1, 2 or 3.

2. A compound according to claim 1 represented by formulae TI: 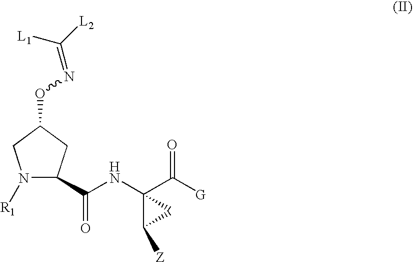
where R₁, G, L₁, L₂ and Z are as previously defined.

3. A compound according to claim 1 represented by formulae III: 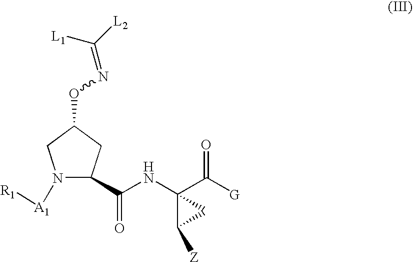
where A₁ is selected from —CO—, —SO₂—; where R₁, G, L₁, L₂ and Z are as previously defined.

4. A compound according to claim 1 represented by formulae IV: 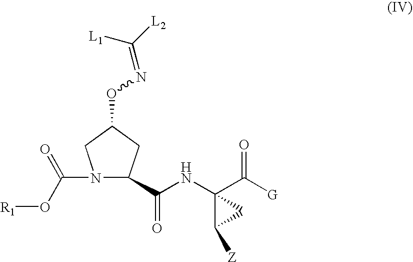
where R₁, G, L₁, L₂ and Z are as previously defined.

5. A compound according to claim 1 represented by formulae V: 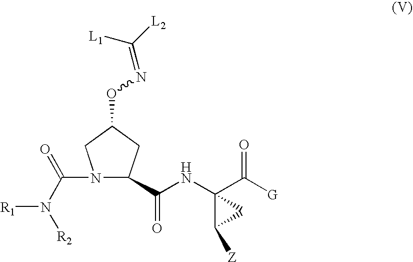
where R₁, R₂, G, L₁, L₂ and Z are as previously defined.

6. A compound according to claim 1 represented by formulae VI: 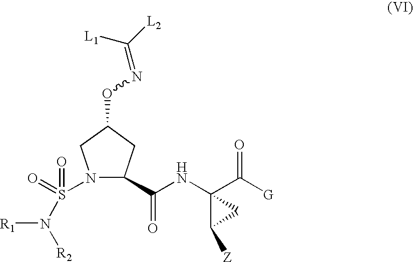
where R₁, R₂, G, L₁, L₂ and Z are as previously defined.

7. A compound according to claim 1 represented by formulae VII: 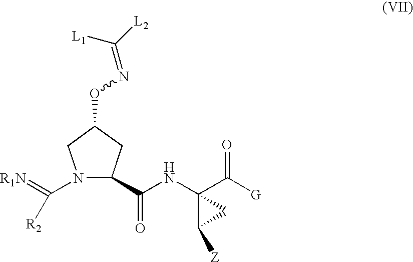
where R₁, R₂, G, L₁, L₂ and Z are as previously defined.

8. A compound according to claim 1 represented by formulae VIII: 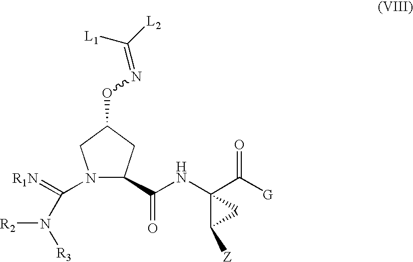
where R₁, R₂, R₃, G, L₁, L₂ and Z are as previously defined.

9. A compound according to claim 1 represented by formulae IX: 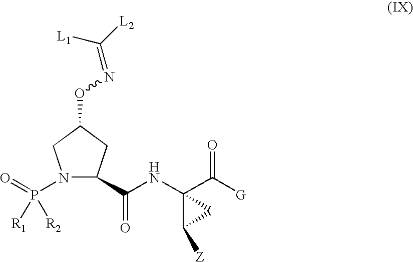
where R₁, R₂, G, L₁, L₂ and Z are as previously defined.

10. A compound according to claim 1 represented by formulae X: 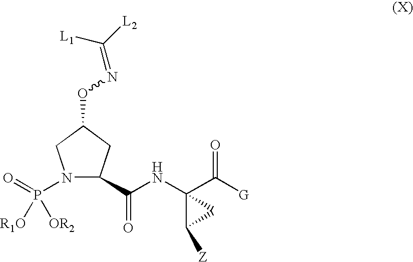
where R₁, R₂, G, L₁, L₂ and Z are as previously defined.

11. A compound according to claim 1 represented by formulae XI: 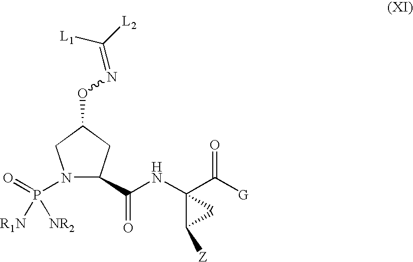
where R₁, R₂, G, L₁, L₂ and Z are as previously defined.

12. A compound of claim 1 having the Formula XII selected from compounds (1)-(160) of Table 1: 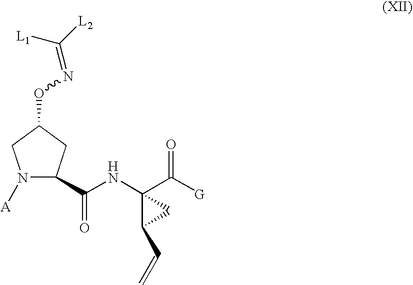
where L₁, L₂, A and G are delineated for each example in TABLE 1:
TABLE 1

| Example | A | L₁L₂ | G |
|---|---|---|---|
| 1 | 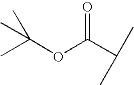 | 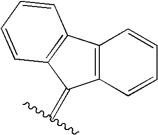 | 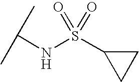 |
| 2 | 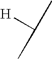 | 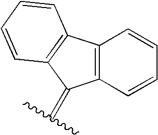 | 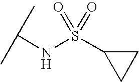 |
| 3 | 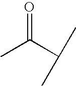 | 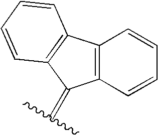 | 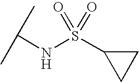 |
| 4 | 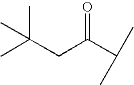 | 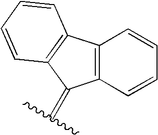 | 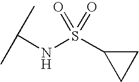 |
| 5 | 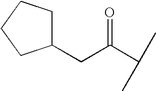 | 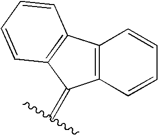 | 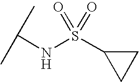 |
| 6 | 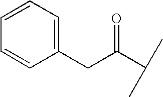 | 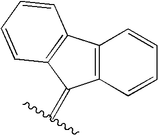 | 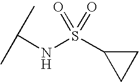 |
| 7 | 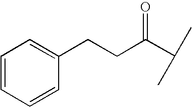 | 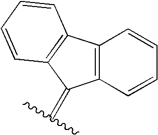 | 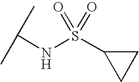 |
| 8 | 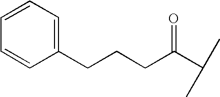 | 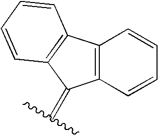 | 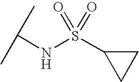 |
| 9 | 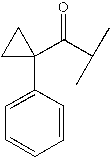 | 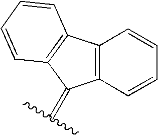 | 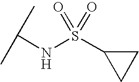 |
| 10 | 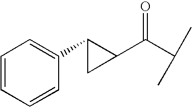 | 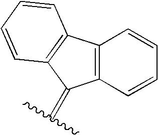 | 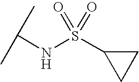 |
| 11 | 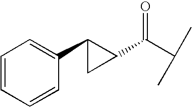 | 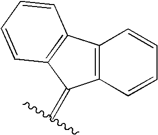 | 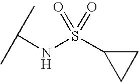 |
| 12 | 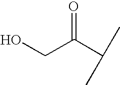 | 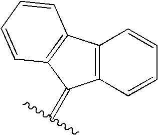 | 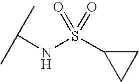 |
| 13 | 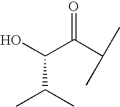 | 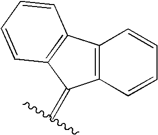 | 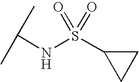 |
| 14 | 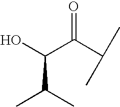 | 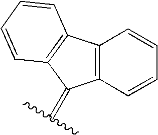 | 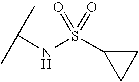 |
| 15 | 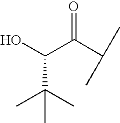 | 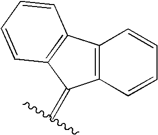 | 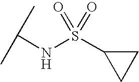 |
| | | | |
|---|---|---|---|
| 16 | 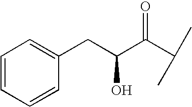 | 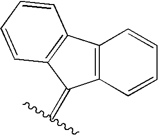 | 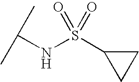 |
| 17 | 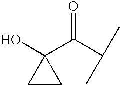 | 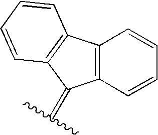 | 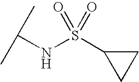 |
| 18 | 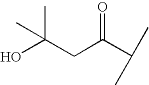 | 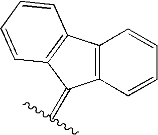 | 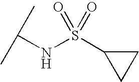 |
| 19 | 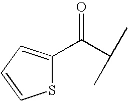 | 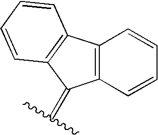 | 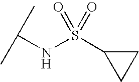 |
| 20 | 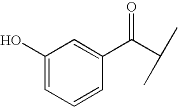 | 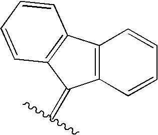 | 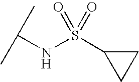 |
| 21 | 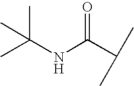 | 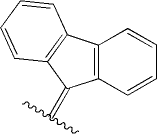 | 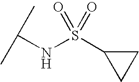 |
| 22 | 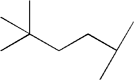 | 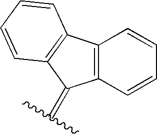 | 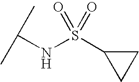 |
| 23 | 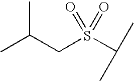 | 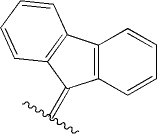 | 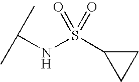 |
| 24 | 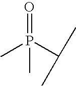 | 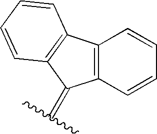 | 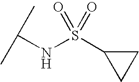 |
| 25 | 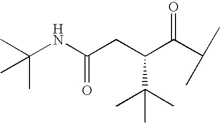 | 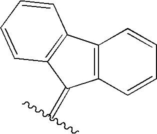 | 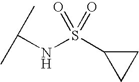 |
| 26 | 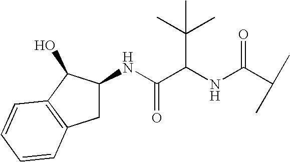 | 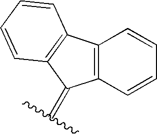 | 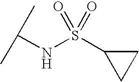 |
| 27 | 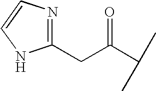 | 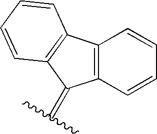 | 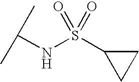 |
| 28 | 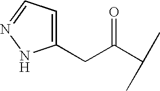 | 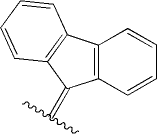 | 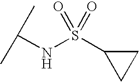 |
| 29 | 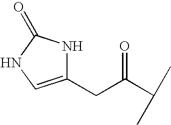 | 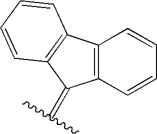 | 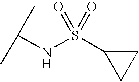 |
| 30 | 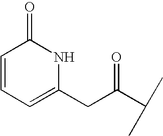 | 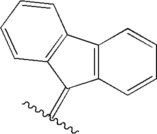 | 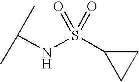 |
| 31 | 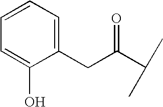 | 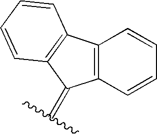 | 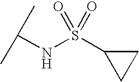 |
| 32 | 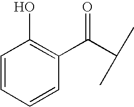 | 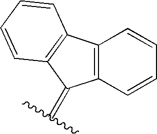 | 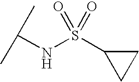 |
| 33 | 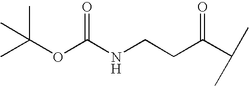 | 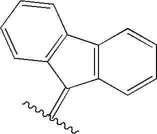 | 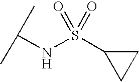 |
| 34 | 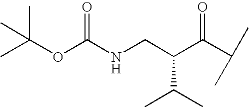 | 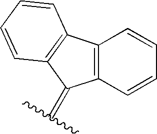 | 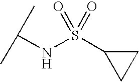 |
| 35 | 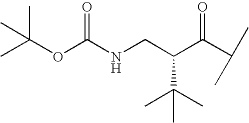 | 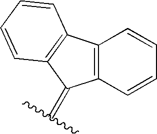 | 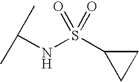 |
| 36 | 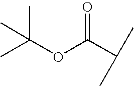 | 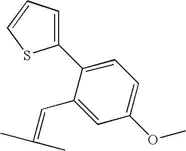 | 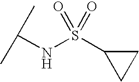 |
| 37 | 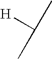 | 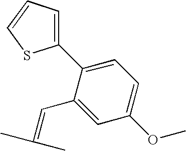 | 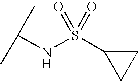 |
| 38 | 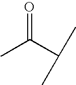 | 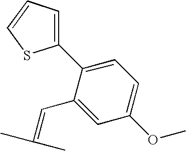 | 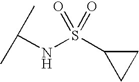 |
| 39 | 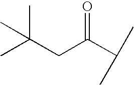 | 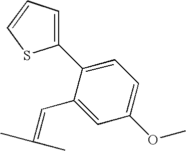 | 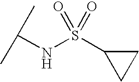 |
| 40 | 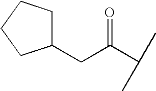 | 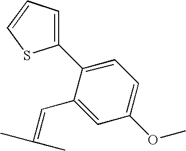 | 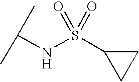 |
| 41 | 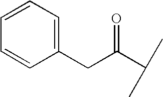 | 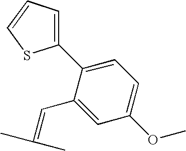 | 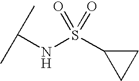 |
| 42 | 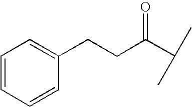 | 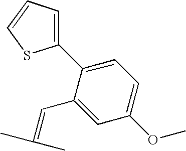 | 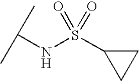 |
| 43 | 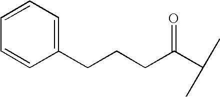 | 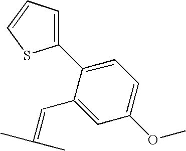 | 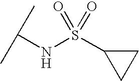 |
| 44 | 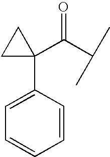 | 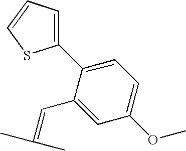 | 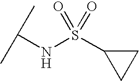 |
| 45 | 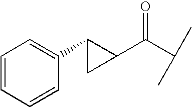 | 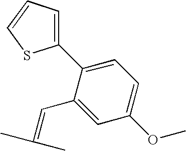 | 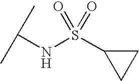 |
| 46 | 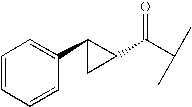 | 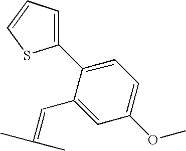 | 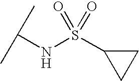 |
| 47 | 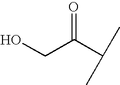 | 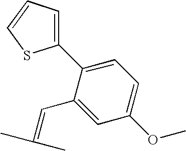 | 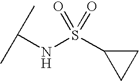 |
| 48 | 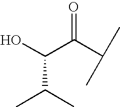 | 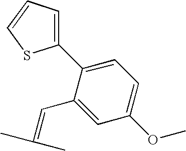 | 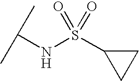 |
| 49 | 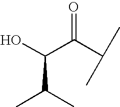 | 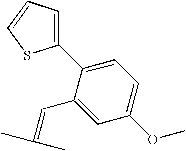 | 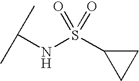 |
| 50 | 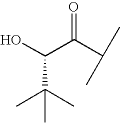 | 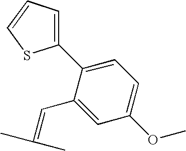 | 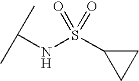 |
| 51 | 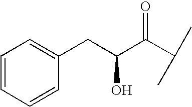 | 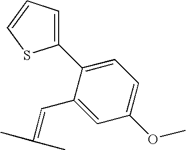 | 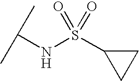 |
| 52 | 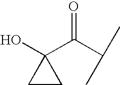 | 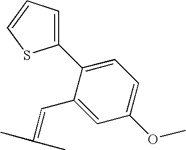 | 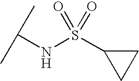 |
| 53 | 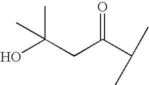 | 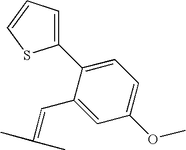 | 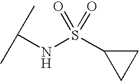 |
| 54 | 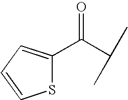 | 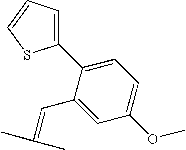 | 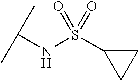 |
| 55 | 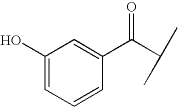 | 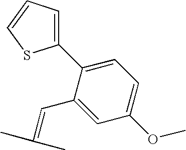 | 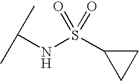 |
| 56 | 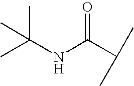 | 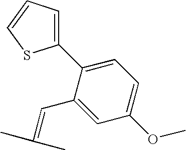 | 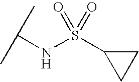 |
| 57 | 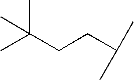 | 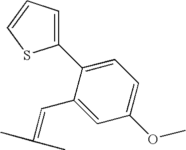 | 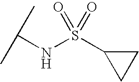 |
| 58 | 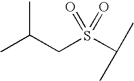 | 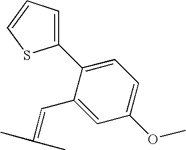 | 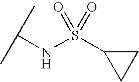 |
| 59 | 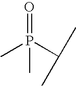 | 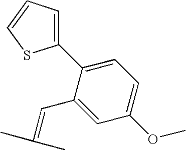 | 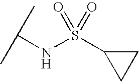 |
| 60 | 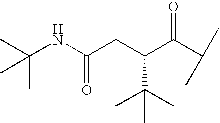 | 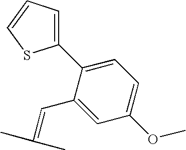 | 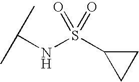 |
| 61 | 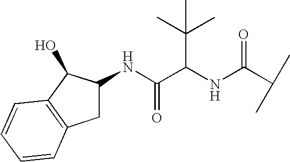 | 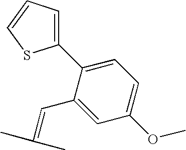 | 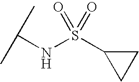 |
| 62 | 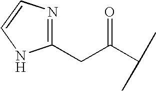 | 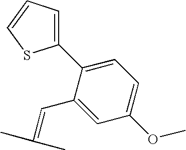 | 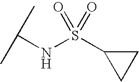 |
| 63 | 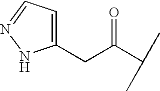 | 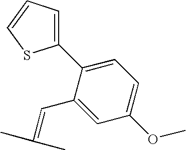 | 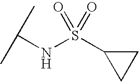 |
| 64 | 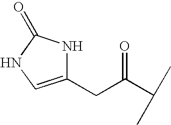 | 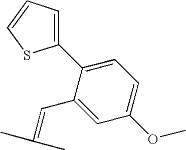 | 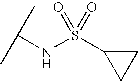 |
| 65 | 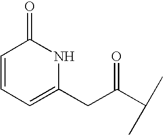 | 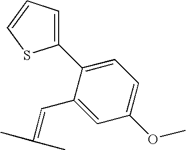 | 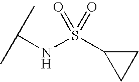 |
| 66 | 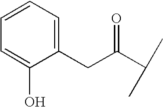 | 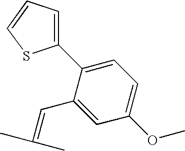 | 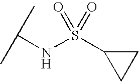 |
| 67 | 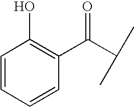 | 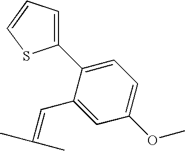 | 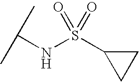 |
| 68 | 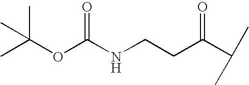 | 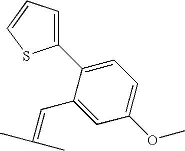 | 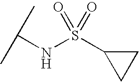 |
| 69 | 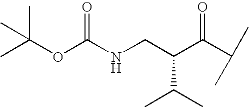 | 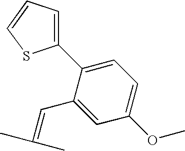 | 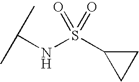 |
| 70 | 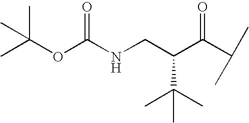 | 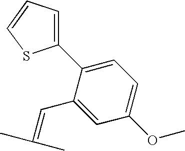 | 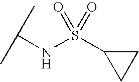 |
| 71 | 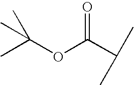 | 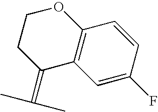 | 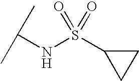 |
| 72 | 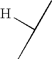 | 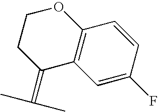 | 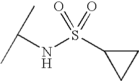 |
| 73 | 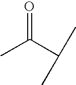 | 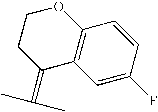 | 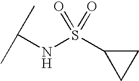 |
| 74 | 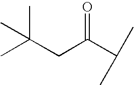 | 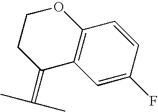 | 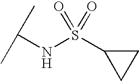 |
| 75 | 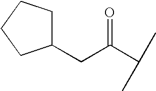 | 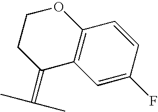 | 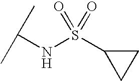 |
| 76 | 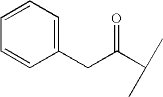 | 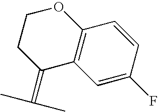 | 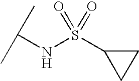 |
| 77 | 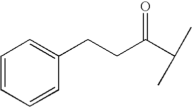 | 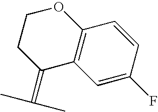 | 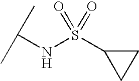 |
| 78 | 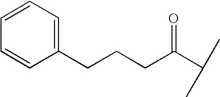 | 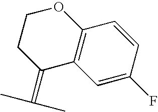 | 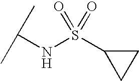 |
| 79 | 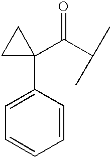 | 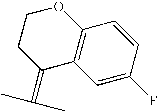 | 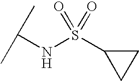 |
| 80 | 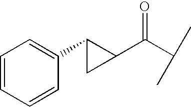 | 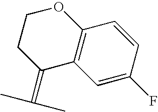 | 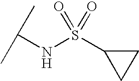 |
| 81 | 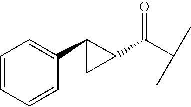 | 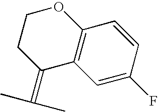 | 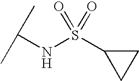 |
| 82 | 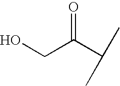 | 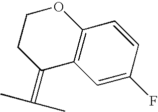 | 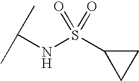 |
| 83 | 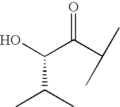 | 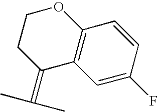 | 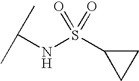 |
| 84 | 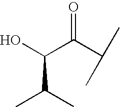 | 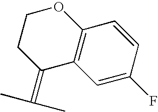 | 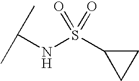 |
| 85 | 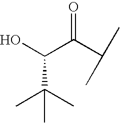 | 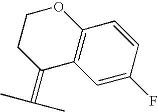 | 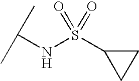 |
| 86 | 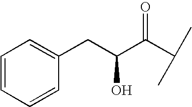 | 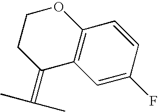 | 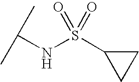 |
| 87 | 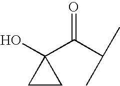 | 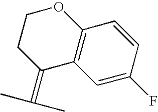 | 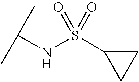 |
| 88 | 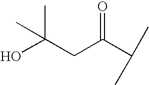 | 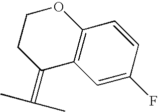 | 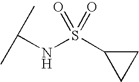 |
| 89 | 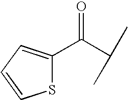 | 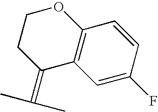 | 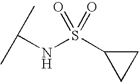 |
| 90 | 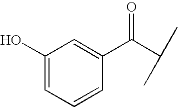 | 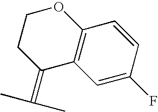 | 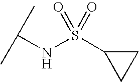 |
| 91 | 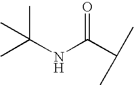 | 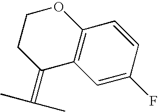 | 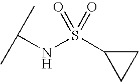 |
| 92 | 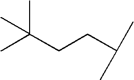 | 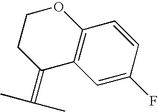 | 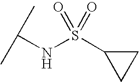 |
| 93 | 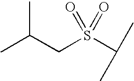 | 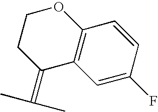 | 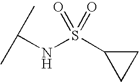 |
| 94 | 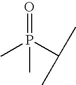 | 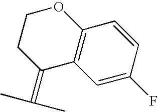 | 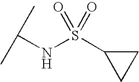 |
| 95 | 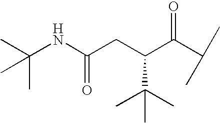 | 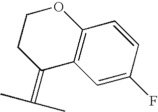 | 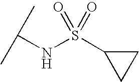 |
| 96 | 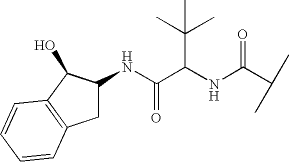 | 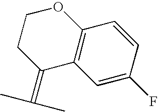 | 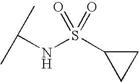 |
| 97 | 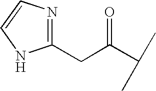 | 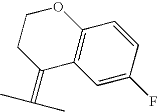 | 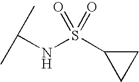 |
| 98 | 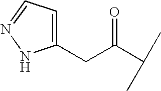 | 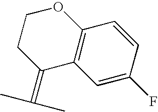 | 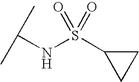 |
| 99 | 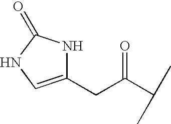 | 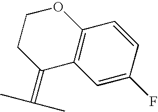 | 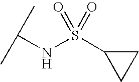 |
| 100 | 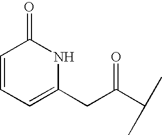 | 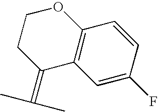 | 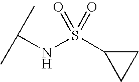 |
| 101 | 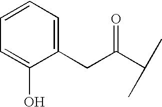 | 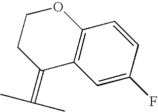 | 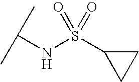 |
| 102 | 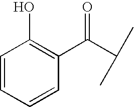 | 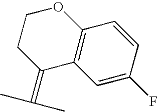 | 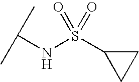 |
| 103 | 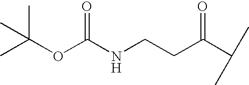 | 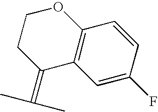 | 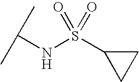 |
| 104 | 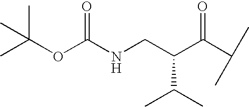 | 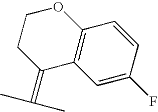 | 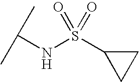 |
| 105 | 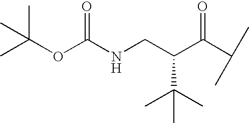 | 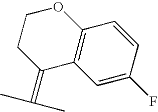 | 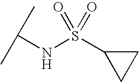 |
| 106 | 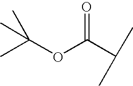 | 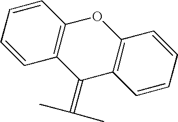 | 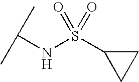 |
| 107 | 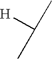 | 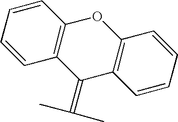 | 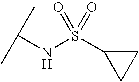 |
| 108 | 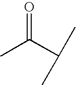 | 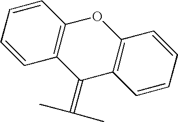 | 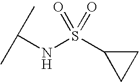 |
| 109 | 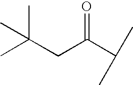 | 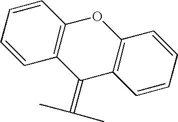 | 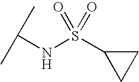 |
| 110 | 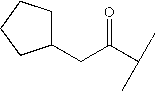 | 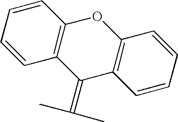 | 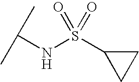 |
| 111 | 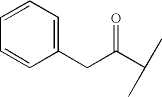 | 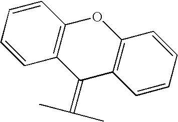 | 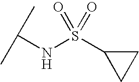 |
| 112 | 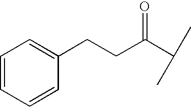 | 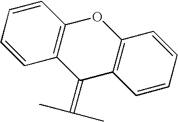 | 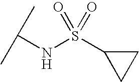 |
| 113 | 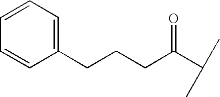 | 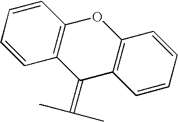 | 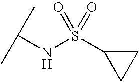 |
| 114 | 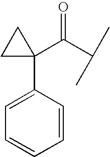 | 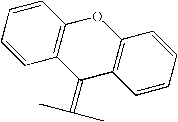 | 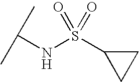 |
| 115 | 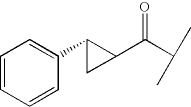 | 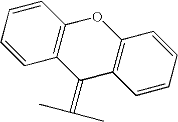 | 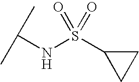 |
| 116 | 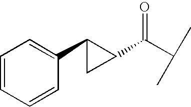 | 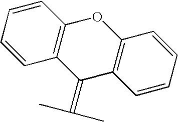 | 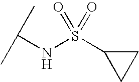 |
| 117 | 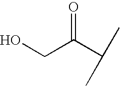 | 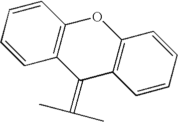 | 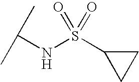 |
| 118 | 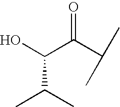 | 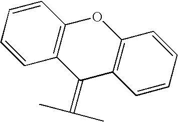 | 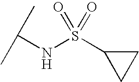 |
| 119 | 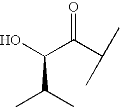 | 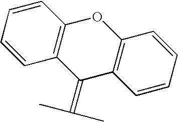 | 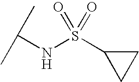 |
| 120 | 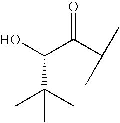 | 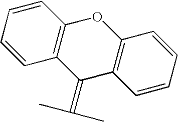 | 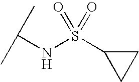 |
| 121 | 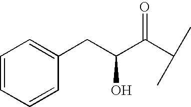 | 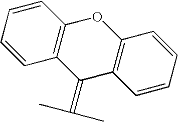 | 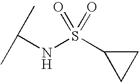 |
| 122 | 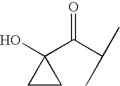 | 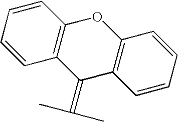 | 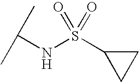 |
| 123 | 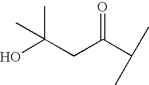 | 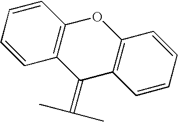 | 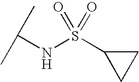 |
| 124 | 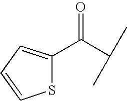 | 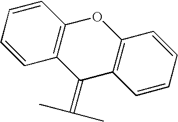 | 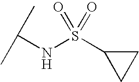 |
| 125 | 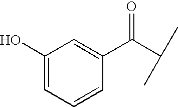 | 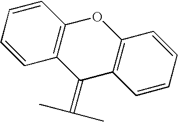 | 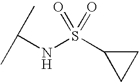 |
| 126 | 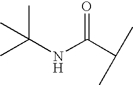 | 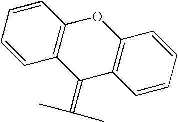 | 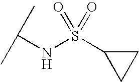 |
| 127 | 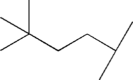 | 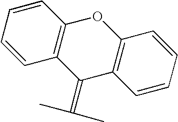 | 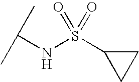 |
| 128 | 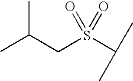 | 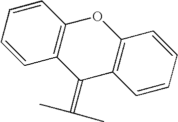 | 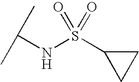 |
| 129 | 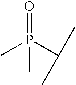 | 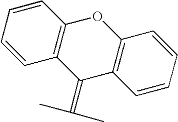 | 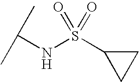 |
| 130 | 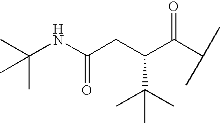 | 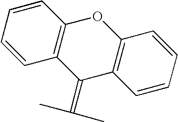 | 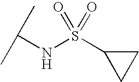 |
| 131 | 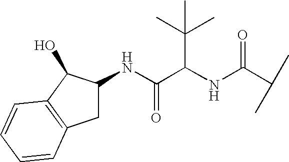 | 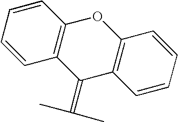 | 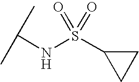 |
| 132 | 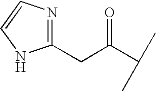 | 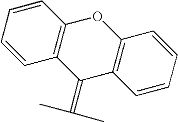 | 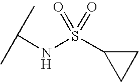 |
| 133 | 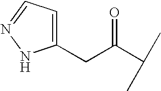 | 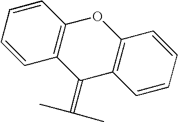 | 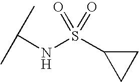 |
| 134 | 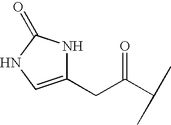 | 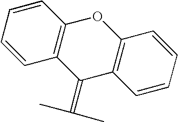 | 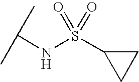 |
| 135 | 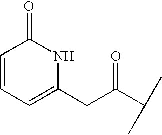 | 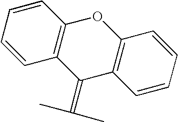 | 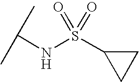 |
| 136 | 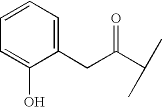 | 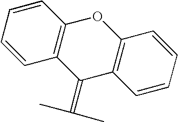 | 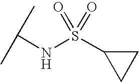 |
| 137 | 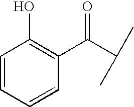 | 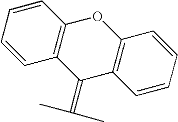 | 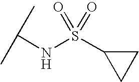 |
| 138 | 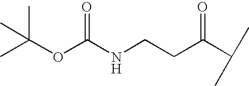 | 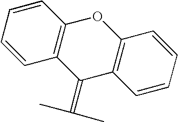 | 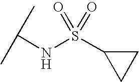 |
| 139 | 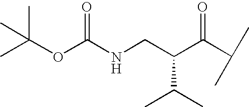 | 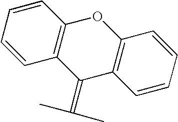 | 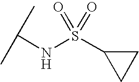 |
| 140 | 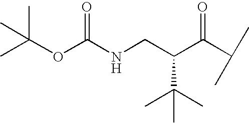 | 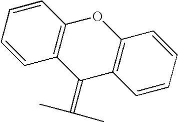 | 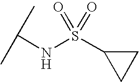 |
| 141 | 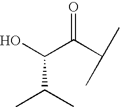 | 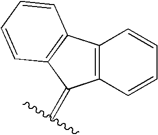 | 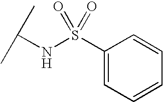 |
| 142 | 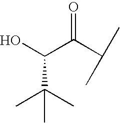 | 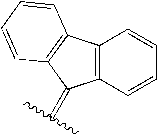 | 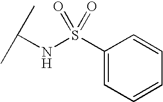 |
| 143 | 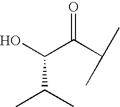 | 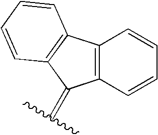 | 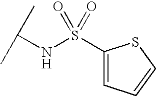 |
| 144 | 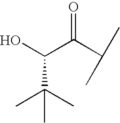 | 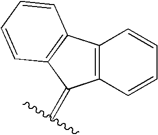 | 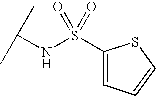 |
| 145 | 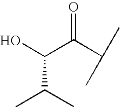 | 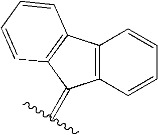 | 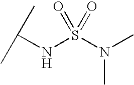 |
| 146 | 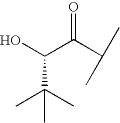 | 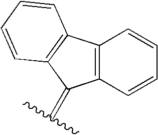 | 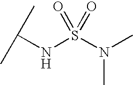 |
| 147 | 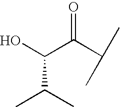 | 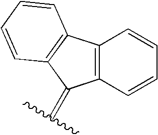 | 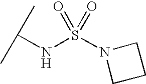 |
| 148 | 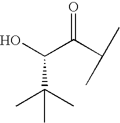 | 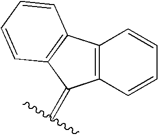 | 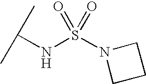 |
| 149 | 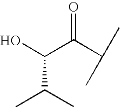 | 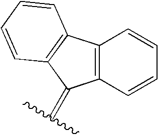 | 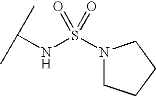 |
| 150 | 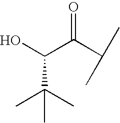 | 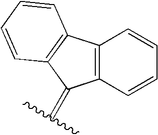 | 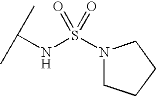 |
| 151 | 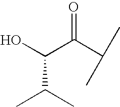 | 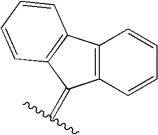 | 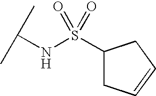 |
| 152 | 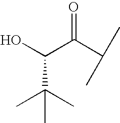 | 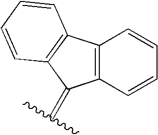 | 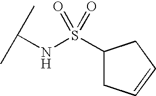 |
| 153 | 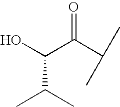 | 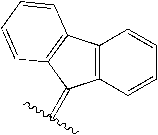 | 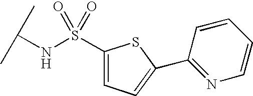 |
| 154 | 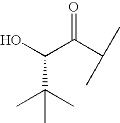 | 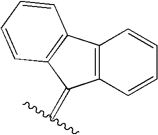 | 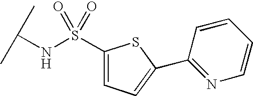 |
| 155 | 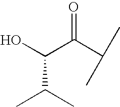 | 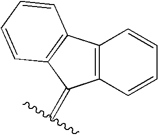 | 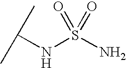 |
| 156 | 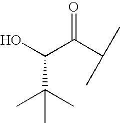 | 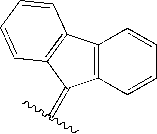 | 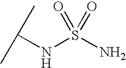 |
| 157 | 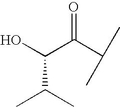 | 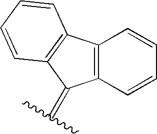 | 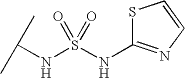 |
| 158 | 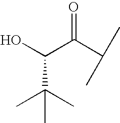 | 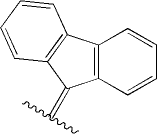 | 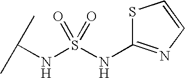 |
| 159 | 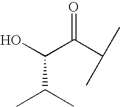 | 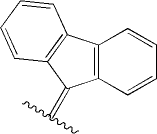 | 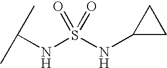 |
| 160 | 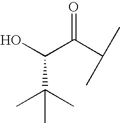 | 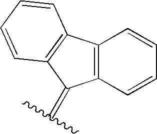 | 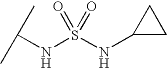 |

13. A pharmaceutical composition comprising an anti-hepatitis C virally effective amount of a compound according to claim 1 or a pharmaceutically acceptable salt, ester, or prodrug thereof, in combination with a pharmaceutically acceptable carrier or excipient.

14. A method of treating a hepatitis C viral infection in a subject, comprising administering to the subject an anti-hepatitis C virally effective amount of a pharmaceutical composition according to claim 13.

15. A method of inhibiting the replication of hepatitis C virus, the method comprising supplying a hepatitis C viral NS3 protease inhibitory amount of the pharmaceutical composition of claim 13.

16. A method of claim 14 further comprising administering concurrently an additional anti-hepatitis C virus agent.

17. The method of claim 16, wherein said additional anti-hepatitis C virus agent is selected from the group consisting of α-interferon, β-interferon, ribavarin, and adamantine.

18. The method of claim 16, wherein said additional anti-hepatitis C virus agent is an inhibitor of other targets in the hepatitis C virus life cycle which is selected from the group consisting of helicase, polymerase, metal loprotease, and IRES.

19. A compound of claim 3 wherein R₁ is:
(1) hydrogen; or
(2) selected from structures (1)-(10): 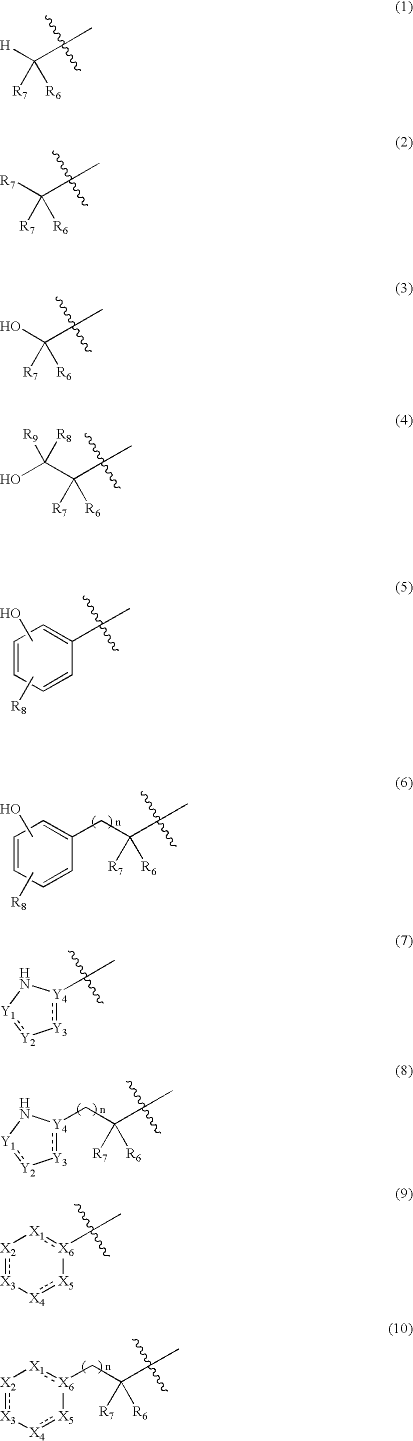
where R₆, R₇, R₈, R₉ are independently selected from the group consisting of:
a) hydrogen;
b) aryl;
c) substituted aryl;
d) heteroaryl;
e) substituted heteroaryl;
f) heterocyclic or substituted heterocyclic;
g) —C₁-C₈ alkyl, —C₂-C₈ alkenyl, or —C₂-C₈ alkynyl containing 0, 1, 2, or 3 heteroatoms selected from O, S or N;
h) substituted —C₁-C₈ alkyl, substituted —C₂-C₈ alkenyl, or substituted —C₂-C₈ alkynyl containing 0, 1, 2, or 3 heteroatoms selected from O, S or N;
i) —C₃-C₁₂ cycloalkyl;
j) substituted —C₃-C₁₂ cycloalkyl;
k) —C₃-C₁₂ cycloalkenyl;
l) substituted —C₃-C₁₂ cycloalkenyl;
or R₆ and R₇ taken together with the carbon atom to which they are attached form a cyclic moiety;
where X₁-X₅ are independently selected from —CO—, —CH—, —NH—, —O— and —N—;
there's at least one —NH— among X₁-X₅; X₆ is selected from —C—, —CH—, —N—; X₁-X₅ can be further substituted when it is a CH or NH; and
where Y₁-Y₃ are independently selected from CO, CH, NH, N, S and O; and Y₁-Y₃ can be further substituted when it is CH or NH; Y₄ is selected from C, CH and N; n=0, 1, 2.

20. Pharmaceutical composition of claim 8 further comprising an additional anti-hepatitis C virus agent.

21. A pharmaceutical composition of claim 20 wherein said additional anti-hepatitis C virus agent is selected from the group consisting of: α-interferon, 1-interferon, ribavarin, and adamantine.

22. Compound of claim 1 wherein said compound is in a substantially pure form.


SEQUENCE LISTING (3 sequences)

SEQ ID NO: 1 - DNA, 18 bp, Artificial Sequence
  Forward Primer
gctgcggcctgtcgagct

SEQ ID NO: 2 - DNA, 19 bp, Artificial Sequence
  Reverse Primer
caaggtcgtctccgcatac

SEQ ID NO: 3 - DNA, 25 bp, Artificial Sequence
  Probe
cgaagctccaggactgcacgatgct